(19) United States
(12) Patent Application Publication
(10) Pub. No.: US 2026/0258336 A1
(43) Pub. Date: Sep. 3, 2026

(54) MICROENVIRONMENT-SIMULATED CELL CULTURE SYSTEM AND MICROENVIRONMENT-SIMULATED CELL CULTURING METHOD

(71) Applicant: FLUIDICONIC BIOTECHNOLOGY CO., LTD., Hsinchu City (TW)

(72) Inventors: Hsuan-Yu MU, Hsinchu City (TW); Jen-Huang HUANG, Hsinchu (TW); Yun Chen LIAN, Hsinchu City (TW); Pei Ching YANG, Hsinchu City (TW); Ya Hui LIN, Hsinchu City (TW)

(21) Appl. No.: 19/653,680

(22) Filed: Apr. 21, 2026

Related U.S. Application Data
(63) Continuation-in-part of application No. 18/061,322, filed on Dec. 2, 2022.

(30) Foreign Application Priority Data
Jun. 30, 2022 (TW) 111124575

(51) Int. Cl. C12M 3/06; C12M 1/00; C12M 1/34; C12N 5/077; C12N 5/078; C12N 5/09

(52) U.S. Cl. CPC C12M 23/16; C12M 23/34; C12M 29/14; C12M 41/40; C12N 5/0634; C12N 5/0656; C12N 5/0693

(57) ABSTRACT

A microenvironment-simulated cell culture system includes a cell culture chip, a fluid storage device and a fluid driving member. The cell culture chip includes a mainbody, a cell culture chamber, two fluid delivery ports and a sample loading well. The cell culture chamber is disposed in the mainbody and includes a first side portion and a second side portion. The two fluid delivery ports are separately disposed on the mainbody and respectively connected to the cell culture chamber. The sample loading well is disposed on the mainbody and connected to the cell culture chamber. The fluid storage device is pipe-connected to the cell culture chip. The fluid driving member is pipe-connected to the fluid storage device and the cell culture chip.

### RELATED APPLICATIONS

This application is a Continuation-in-part of U.S. application Ser. No. 18/061,322, filed on Dec. 2, 2022, which claims priority to Taiwan Application Serial Number 111124575, filed Jun. 30, 2022, which is herein incorporated by reference.

### BACKGROUND

### Technical Field

The present disclosure relates to a cell culture system and a cell culture method. More particularly, the present disclosure relates to microenvironment-simulated cell culture system and a microenvironment-simulated cell culturing method which can simulate the microenvironment during cell growth.

### Description of Related Art

In the modern society, cancer has brought a great threat to the safety of human life, and thus how to effectively detect cancer early and take the appropriate treatment are important research goals in the current clinical practice.

The screening of anticancer drugs is mostly achieved by two-dimensional cell culture, three-dimensional cell spheroid culture or animal experiments. However, in the two-dimensional cell culture, the growth of the cancer cells can only be observed on surfaces, and it cannot represent the actual complexity of the tumor microenvironment. Further, the characteristics of the cell diversity and the richness of the extracellular matrix in tumors also cannot be simulated, resulting in the drug screening results are not consistent with the actual pharmacological effects. Furthermore, in the three-dimensional cell spheroid culture, although the tissue characteristics in tumors can be simulated, but the gradient of oxygen, the nutrient distribution and the distribution of immune cells are not easy to observe in the cell spheroid. Moreover, by performing animal experiments, the time and the cost for screening anticancer drugs may be extremely increased, and the reproducibility of the experiment is not as good as expected.

Therefore, how to improve the in vitro cell culture device so as to accurately simulate the microenvironment of cell growth in tumors and then screen anticancer drugs for different types of cancer or develop new treatment guidelines has become the aim of modern practitioners and academics.

### SUMMARY

According to one aspect of the present disclosure, a microenvironment-simulated cell culture system includes a cell culture chip, a fluid storage device and a fluid driving member. The cell culture chip includes a mainbody, a cell culture chamber, two fluid delivery ports and a sample loading well. The cell culture chamber is disposed in the mainbody and includes a first side portion and a second side portion, wherein the first side portion and the second side portion are respectively disposed on two ends of the cell culture chamber along a long axis of the mainbody. The two fluid delivery ports are separately disposed on the mainbody and respectively connected to the cell culture chamber. The sample loading well is disposed on the mainbody and connected to the cell culture chamber. The fluid storage device is pipe-connected to the cell culture chip, wherein the fluid storage device is connected to the cell culture chamber by one of the fluid delivery ports. The fluid driving member is pipe-connected to the fluid storage device and the cell culture chip, wherein the fluid driving member is connected to the cell culture chamber by the other one of the fluid delivery ports. The cell culture chamber is substantially a long-stripped slot, two long sides of the cell culture chamber are parallel to the long axis of the mainbody, a length ratio of a short side of the cell culture chamber to one of the long sides of the cell culture chamber is 1:1 to 1:4, and a length of the one of the long sides of the cell culture chamber is 1.3 cm to 2.0 cm.

According to another aspect of the present disclosure, a microenvironment-simulated cell culture system includes a cell culture chip, a fluid storage device and an air pump. The cell culture chip includes at least one cell culture chamber, at least one fluid storing well, at least three membrane pump units, at least one air transporting channel, at least one valve and at least two air transporting pores. The at least one cell culture chamber includes a first side portion and a second side portion, and the first side portion and the second side portion are respectively disposed on two ends of the at least one cell culture chamber along a long axis of the cell culture chip. The at least one fluid storing well is connected to the at least one cell culture chamber. The at least three membrane pump units are connected to the at least one fluid storing well, wherein the at least three membrane pump units are arranged in sequence. The at least one air transporting channel is connected among the at least three membrane pump units, wherein the at least one air transporting channel is substantially s-shaped, and a width of the air transporting channel is 0.06 mm to 0.12 mm. The at least one valve is connected to the air transporting channel. One of the at least two air transporting pores is connected to the at least three membrane pump units, and the other one of the at least two air transporting pores is connected to the at least one valve. The fluid storage device is connected to the at least one fluid storing well, wherein the fluid storage device is for supplying a cell culture medium to the at least one fluid storing well. The air pump is connected to the at least two air transporting pores, wherein the at least one valve is connected between the air transporting channel and the air pump. The at least one cell culture chamber is substantially a long-stripped slot, two long sides of the at least one cell culture chamber are parallel to the long axis of the cell culture chip, a length ratio of a short side of the at least one cell culture chamber to one of the long sides of the at least one cell culture chamber is 1:1 to 1:4, and a length of the one of the long sides of the at least one cell culture chamber is 1.3 cm to 2.0 cm.

According to another aspect of the present disclosure, a microenvironment-simulated cell culturing method includes following steps. The microenvironment-simulated cell culture system is provided. A cell is seeded in the at least one cell culture chamber via the at least one fluid storing well, wherein the air pump drives the at least three membrane pump units in sequence to transport the cell culture medium including the cell in the at least one fluid storing well to the at least one cell culture chamber, and a number of the cell seeded in the at least one cell culture chamber is 0.25×10⁶ to 2.5×10⁷. The cell is incubated for a reaction time and then a biochemical condition of the cell is detected so as to obtain a microenvironment-simulated cell culturing result. A positive pressure of the air pump is 3 to 12 psi, a negative pressure of the air pump is −3 to −12 psi, and a flow rate of the cell culture medium driven by the air pump is 30 μL/min to 60 μL/min.

### BRIEF DESCRIPTION OF THE DRAWINGS

The present disclosure can be more fully understood by reading the following detailed description of the embodiment, with reference made to the accompanying drawings as follows:

FIG. 1 is a schematic view of a microenvironment-simulated cell culture system according to one embodiment of the present disclosure.

FIG. 2 is a schematic view of a cell culture chip of the microenvironment-simulated cell culture system of FIG. 1.

FIG. 3 is an exploded view of the cell culture chip of FIG. 2.

FIG. 4 is a schematic view of a microenvironment-simulated cell culture system according to another embodiment of the present disclosure.

FIG. 5 is an exploded view of the cell culture chip of FIG. 4.

FIG. 6 is a flow chart of a microenvironment-simulated cell culturing method according to another embodiment of the present disclosure.

FIG. 7 shows the results of Western blotting analysis of the co-cultivation of 4T1 cells and K-BALB cells for 24 hours by the microenvironment-simulated cell culture systems of Example 1 to Example 3.

FIG. 8 shows the analysis results of hypoxia signal quantification in different areas of the cell culture chamber of the microenvironment simulated cell culture system of Example 2.

FIG. 9 shows the analysis results of cell viability in different areas of the cell culture chamber after culturing for 24 hours with different drug combinations.

FIG. 10A shows the staining results of the propidium iodide in different areas of the cell culture chamber of Testing example 1.

FIG. 10B shows the staining results of the propidium iodide in Area 4 of the cell culture chambers of Testing example 1 to Testing example 4.

FIG. 11 shows the quantification results of mRNA expression in the normoxia area and the hypoxia area of the cell culture chamber of Testing example 1.

FIG. 12A shows the quantification results of mRNA expression in the normoxia areas of the cell culture chambers of Testing example 1 and Testing example 3.

FIG. 12B shows the quantification results of mRNA expression in the hypoxia areas of the cell culture chambers of Testing example 1 and Testing example 3.

FIG. 13A shows the quantification results of mRNA expression of the K-BALB cells in the normoxia areas of the cell culture chambers of Control group 2 and Testing example 4 to Testing example 6.

FIG. 13B shows the quantification results of mRNA expression of the K-BALB cells in the hypoxia areas of the cell culture chambers of Control group 2 and Testing example 4 to Testing example 6.

FIG. 14A shows the quantification results of mRNA expression of 4T1 cells in the normoxia areas of the cell culture chambers of Control group 3, Testing example 7 and Testing example 8.

FIG. 14B shows the quantification results of mRNA expression of 4T1 cells in the hypoxia areas of the cell culture chambers of Control group 3, Testing example 7 and Testing example 8.

FIG. 15A shows the analysis results of the percentage of T cells expressing Tim-3 receptor in the normoxia area and the hypoxia area of the cell culture chamber.

FIG. 15B shows the analysis results of the percentage of T cells expressing CTLA-4 receptor in the normoxia area and the hypoxia area of the cell culture chamber.

FIG. 15C shows the analysis results of the percentage of T cells expressing PD-1 receptor in the normoxia area and the hypoxia area of the cell culture chamber.

FIG. 16 shows the staining results of T cells in different areas of the cell culture chamber after being cultured for 4 hours and 24 hours.

FIG. 17 shows the staining results of apoptosis signaling-related proteins in different areas of the cell culture chamber.

FIG. 18A shows the analysis results of the ratio of T cells to the total cell mass in the hypoxia area of the cell culture chamber after being treated with different doses of anti-PD-1 antibody.

FIG. 18B shows the analysis results of the ratio of T cells to the total cell mass in the normoxia area of the cell culture chamber after being treated with different doses of anti-PD-1 antibody.

FIG. 19A shows the analysis results of the percentage of apoptosis in the hypoxia area of the cell culture chamber after being treated with different doses of anti-PD-1 antibody.

FIG. 19B shows the analysis results of the percentage of apoptosis in the normoxia area of the cell culture chamber after being treated with different doses of anti-PD-1 antibody.

FIG. 20A shows the analysis results of the ratio of T cells to the total cell mass in the hypoxia area of the cell culture chamber after being treated with different drugs.

FIG. 20B shows the analysis results of the ratio of T cells to the total cell mass in the normoxia area of the cell culture chamber after being treated with different drugs.

FIG. 21A shows the analysis results of the percentage of apoptosis in the hypoxia area of the cell culture chamber after being treated with different drugs.

FIG. 21B shows the analysis results of the percentage of apoptosis in the normoxia area of the cell culture chamber after being treated with different drugs.

FIG. 22 shows an image of the 4T1 cells cultured in the cell culture chamber of the microenvironment-simulated cell culture system of Example 4.

FIG. 23A shows the diffusion result of oxygen in the cell culture chamber of the microenvironment-simulated cell culture system of Example 4 on the static condition and the dynamic condition.

FIG. 23B shows the diffusion result of micromolecule in the cell culture chamber of the microenvironment-simulated cell culture system of Example 4 on the static condition and the dynamic condition.

FIG. 23C shows the diffusion result of macromolecule in the cell culture chamber of the microenvironment-simulated cell culture system of Example 4 on the static condition and the dynamic condition.

FIG. 24A shows the analysis results of apoptosis of the 4T1 cells treated with different concentrations of the doxorubicin for 24 hours.

FIG. 24B shows the analysis results of apoptosis of the 4T1 cells treated with different concentrations of the doxorubicin for 48 hours.

FIG. 24C shows the analysis results of apoptosis of the 4T1 cells treated with different concentrations of the doxorubicin for 72 hours.

FIG. 25A shows the oxygenation profiles of the HEPG2 cells in different areas of the cell culture chamber of the microenvironment-simulated cell culture system of Example 4 with different seeding densities.

FIG. 25B shows the oxygenation profiles of the MIA-PaCa-2 cells in different areas of the cell culture chamber of the microenvironment-simulated cell culture system of Example 4 with different seeding densities.

FIG. 25C shows the oxygenation profiles of the SW620 cells in different areas of the cell culture chamber of the microenvironment-simulated cell culture system of Example 4 with different seeding densities.

FIG. 26A shows the oxygenation profiles of the WiDr cells in different areas of the cell culture chamber of the microenvironment-simulated cell culture system of Example 4 with different seeding densities.

FIG. 26B shows the oxygenation profiles of the HCT-116 cells in different areas of the cell culture chamber of the microenvironment-simulated cell culture system of Example 4 with different seeding densities.

FIG. 26C shows the oxygenation profiles of the MDA-MB-231 cells in different areas of the cell culture chamber of the microenvironment-simulated cell culture system of Example 4 with different seeding densities.

FIG. 27A shows the signals of Caspase 3/7 of the cancer cells of PDAC mice in different areas of the cell culture chamber of the microenvironment-simulated cell culture system of Example 4.

FIG. 27B shows the signals of Caspase 3/7 of the cancer cells of CRC mice in different areas of the cell culture chamber of the microenvironment-simulated cell culture system of Example 4.

FIG. 27C shows the signals of Caspase 3/7 of the cancer cells of BC mice in different areas of the cell culture chamber of the microenvironment-simulated cell culture system of Example 4.

FIG. 28A shows the numbers of the macrophages (CD45⁺, F4/80⁺) in the original the constructed cancer tumor tissue, tissue in the microenvironment-simulated cell culture system of Example 4 and the tumoroid.

FIG. 28B shows the numbers of the CD3⁺/CD8⁺ T-cell in the original tumor tissue, the constructed cancer tissue in the microenvironment-simulated cell culture system of Example 4 and the tumoroid.

FIG. 28C shows the numbers of the CD3⁺/CD4⁺ T-cell in the original tumor tissue, the constructed cancer tissue in the microenvironment-simulated cell culture system of Example 4 and the tumoroid.

FIG. 28D shows the numbers of the cancer cell in the original tumor tissue, the constructed cancer tissue in microenvironment-simulated cell culture system of Example 4 and the tumoroid.

FIG. 29 shows the oxygenation profiles of the AML-12 liver cells in different areas of the cell culture chamber of the microenvironment-simulated cell culture system of Example 4 with different seeding densities.

FIG. 30A shows the dead cell count result of the cancer cells co-cultured with the PBMC in different areas of the cell culture chamber of the microenvironment-simulated cell culture system of Example 4.

FIG. 30B shows the dead cell count result of the cancer cells treated with the pembrolizumab in different areas of the cell culture chamber of the microenvironment-simulated cell culture system of Example 4.

FIG. 30C shows the dead cell count result of the cancer cells co-cultured with 5% PBMC and treated with the pembrolizumab in different areas of the cell culture chamber of the microenvironment-simulated cell culture system of Example 4.

FIG. 30D shows the dead cell count result of the cancer cells co-cultured with 20% PBMC and treated with the pembrolizumab in different areas of the cell culture chamber of the microenvironment-simulated cell culture system of Example 4.

### DETAILED DESCRIPTION

The present disclosure will be further exemplified by the following specific embodiments to facilitate utilizing and practicing the present disclosure completely by the people skilled in the art without over-interpreting and over-experimenting. However, these practical details are used to describe how to implement the materials and methods of the present disclosure and are not necessary.

### [The Microenvironment-Simulated Cell Culture System of the Present Disclosure]

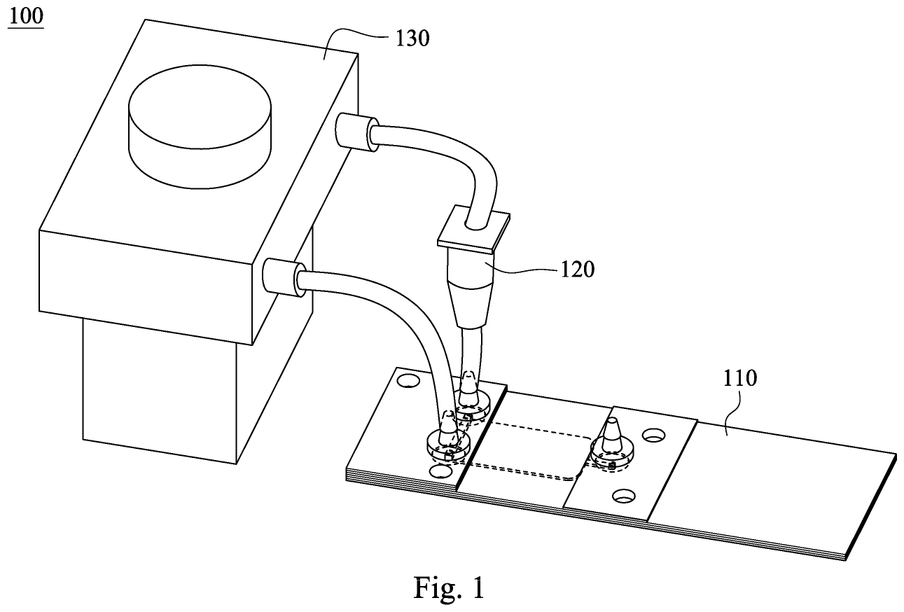

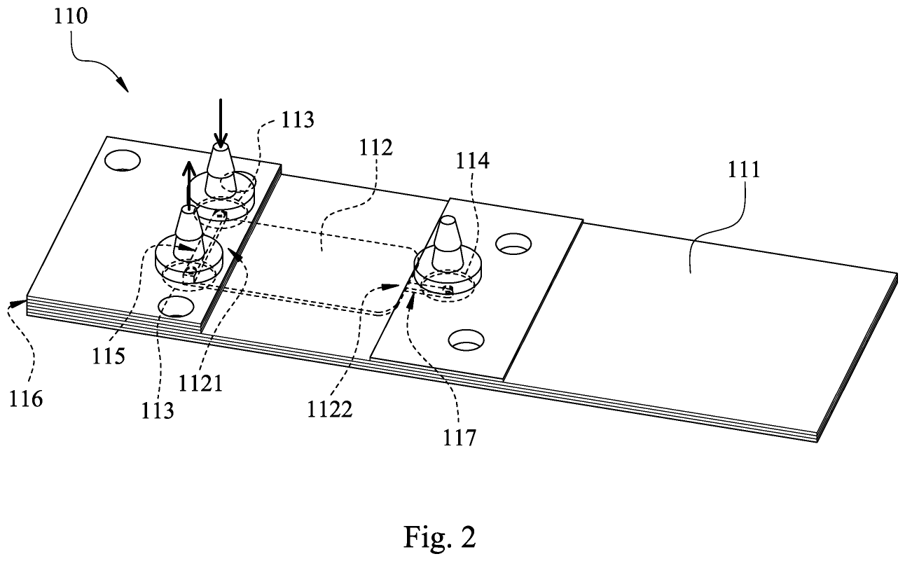

Reference is made to FIG. 1 and FIG. 2, wherein FIG. 1 is a schematic view of a microenvironment-simulated cell culture system 100 according to one embodiment of the present disclosure, and FIG. 2 is a schematic view of a cell culture chip 110 of the microenvironment-simulated cell culture system 100 of FIG. 1. The microenvironment-simulated cell culture system 100 includes a cell culture chip 110, a fluid storage device 120 and a fluid driving member 130.

The cell culture chip 110 includes a mainbody 111, a cell culture chamber 112, two fluid delivery ports 113 and a sample loading well 114.

The cell culture chamber 112 is disposed in the mainbody 111. The cell culture chamber 112 includes a first side portion 1121 and a second side portion 1122, the first side portion 1121 and the second side portion 1122 are respectively disposed on two ends of the cell culture chamber 112 along a long axis (not shown) of the mainbody 111. In particular, in the embodiment of FIG. 1, the mainbody 111 is substantially a rectangle, the cell culture chamber 112 is substantially a long-stripped slot, and the first side portion 1121 and the second side portion 1122 are respectively located in the cell culture chamber 112 and are near two end portions of the mainbody 111. Furthermore, as shown in FIG. 2, two long sides of the cell culture chamber 112 are parallel to the long axis of the mainbody 111, a length ratio of a short side of the cell culture chamber 112 to one of the long sides of the cell culture chamber 112 is 1:1 to 1:4, and a length of the one of the long sides of the cell culture chamber 112 is 1.3 cm to 2.0 cm. Thus, it is favorable for establishing a molecular gradient in the cell culture chamber 112 when the cells are cultured therein. Preferably, the length ratio of the short side of the cell culture chamber 112 to the one of the long sides of the cell culture chamber 112 can be 1:2. Moreover, a height of the cell culture chip 110 can be 0.25 mm to 0.75 mm, and the present disclosure is not limited thereto.

Further, a height of the cell culture chamber 112 perpendicular to the long axis of the mainbody 111 can be 180 μm to 220 μm, and a length of the short side of the cell culture chamber 112 is 0.5 cm to 1.0 cm. Therefore, a direct optical visualization across the full depth of the construct can be achieved.

The two fluid delivery ports 113 are separately disposed on the mainbody 111, and the two fluid delivery ports 113 are respectively connected to the cell culture chamber 112 so as to transport the fluid in the cell culture chamber 112. Further, the two fluid delivery ports 113 can be separately disposed along a direction parallel to the short side of the cell culture chamber 112 so as to establish the fluid circulation subsequently, and the present disclosure is not limited thereto.

The sample loading well 114 is disposed on the mainbody 111, and the sample loading well 114 is connected to the cell culture chamber 112 so as to transport the cells to be cultured into the cell culture chamber 112.

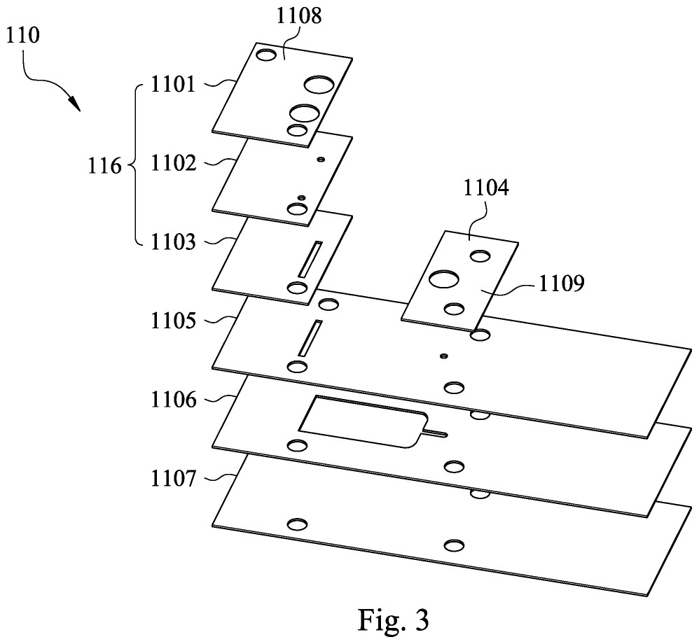

Reference is made to FIG. 2 and FIG. 3 simultaneously, wherein FIG. 3 is an exploded view of the cell culture chip 110 of FIG. 2. As shown in FIG. 3, the cell culture chip 110 is a multi-layers structure and includes a first base plate 1101, a second base plate 1102, a third base plate 1103, a fourth base plate 1104, a fifth base plate 1105, a sixth base plate 1106 and a seventh base plate 1107.

As shown in FIG. 2 and FIG. 3, the first base plate 1101 has a first surface 1108, and the two fluid delivery ports 113 are separately opened on the first surface 1108 (reference number is shown in FIG. 3), wherein the first base plate 1101, the second base plate 1102, the third base plate 1103 and the fifth base plate 1105 are stacked in sequence to form a fluid channel 115 (reference number is shown in FIG. 2), and the fluid channel 115 is connected to the two fluid delivery ports 113 and the cell culture chamber 112. Further, the first base plate 1101, the second base plate 1102 and the third base plate 1103 are stacked in sequence to form a first covering unit 116, and the first covering unit 116 covers the first side portion 1121. Therefore, by the arrangements that the two fluid delivery ports 113 are opened on the first surface 1108 of the first base plate 1101, and the first covering unit 116 formed by the first base plate 1101, the second base plate 1102 and the third base plate 1103 covers the first side portion 1121, the fluid input from one of the fluid delivery ports 113 can be transported into the cell culture chamber 112 by the fluid channel 115 and then transported out of the cell culture chamber 112 by the other one of the fluid delivery ports 113. Therefore, it is favorable for effectively simulating the material exchange between the tumor and the external environment thereof, and the interaction between the tumor microenvironment and blood vessels, such as the shear force and the normal force caused by the flow of the blood and the tissue fluid to the tumor tissue, etc., also can be simulated. Thus, it has excellent clinical application potential.

As shown in FIG. 2 and FIG. 3, the fourth base plate 1104 has a second surface 1109 (reference number is shown in FIG. 3), and the sample loading well 114 is opened on the second surface 1109. The fourth base plate 1104 and the fifth base plate 1105 are stacked in sequence to form a loading channel 117 (reference number is shown in FIG. 2), the loading channel 117 is connected to the sample loading well 114 and the cell culture chamber 112, and the fourth base plate 1104 can cover the second side portion 1122, or be disposed adjacent to the second side portion 1122 to cover the loading channel 117. Therefore, by the arrangements that the sample loading well 114 is opened on the second surface 1109 of the fourth base plate 1104, and the fourth base plate 1104 covers the second side portion 1122 or is disposed adjacent to the second side portion 1122, the suspension including the cells to be cultured can be transported into the cell culture chamber 112 by the sample loading well 114 so as to facilitate the cells to adhere and then grow in the three-dimensional space of the cell culture chamber 112. Further, if the extracellular matrix fluid (such as collagen) is fully mixed with the cell suspension and then transported into the cell culture chamber 112 through the sample loading well 114, not only the cells can grow in the three-dimensional space of the cell culture chamber 112, but also the states that the tumor is rich in the extracellular matrix and the hyperplasia of the connective tissues can be simulated, and the present disclosure is not limited thereto.

Furthermore, as shown in FIG. 2 and FIG. 3, the fifth base plate 1105, the sixth base plate 1106 and the seventh base plate 1107 are stacked in sequence to form the cell culture chamber 112. Therefore, the assembling margin of the cell culture chip 110 can be effectively enhanced, so that the overall structure thereof can be more stable.

Further, the first base plate 1101, the second base plate 1102, the third base plate 1103, the fourth base plate 1104, the fifth base plate 1105, the sixth base plate 1106 and the seventh base plate 1107 can be made of an impermeable material. In particular, by the arrangement that the cell culture chip 110 is formed by stacking a plurality of impermeable base plates, the areas where the cell culture chamber 112 can communicate with a chip-external space are restricted to the two fluid delivery ports 113 on the first side portion 1121 and the sample loading well 114 on the second side portion 1122. Furthermore, after the cells to be cultured are transported to the cell culture chamber 112 by the sample loading well 114, the sample loading well 114 will be closed. At this time, the areas where the substances can be exchanged between the cell culture chamber 112 and the external space are only the two fluid delivery ports 113, so that it is favorable for establishing the molecular gradient along the first side portion 1121 to the second side portion 1122 of the cell culture chamber 112, and the molecular gradient can influence the cell growth during the cultivation of cells subsequently. Furthermore, the impermeable material can be polydimethylsiloxane, poly(methyl methacrylate), polyethylene terephthalate, acrylic, polycarbonate, polystyrene, silicone rubber or glass, but the present disclosure is not limited thereto. Moreover, the impermeable material can be a transparent material so as to facilitate the direct observation thereof and then enhance the convenience of use.

The fluid storage device 120 is pipe-connected to the cell culture chip 110, and the fluid storage device 120 is connected to the cell culture chamber 112 by one of the fluid delivery ports 113.

The fluid driving member 130 is pipe-connected to the fluid storage device 120 and the cell culture chip 110, and the fluid driving member 130 is connected to the cell culture chamber 112 by the other one of the fluid delivery ports 113.

In particular, the fluid storage device 120 and the fluid driving member 130 are respectively connected to the cell culture chamber 112 by different fluid delivery ports 113, and the fluid storage device 120 is for storing a cell culture medium. The fluid driving member 130 is for continuously driving the cell culture medium to be transferred from the fluid storage device 120 to the cell culture chamber 112 through the one of the fluid delivery ports 113 and then be removed from the cell culture chamber 112 through the other one of the fluid delivery ports 113, and the cell culture medium will continue to circulate and flow along this path so as to establish a dynamic fluid circulation system in the cell culture chip 110. Thus, the interaction between tumors and the circulatory system in the organism can be simulated so as to apply in the subsequent applications.

Furthermore, the fluid driving member 130 can be a peristaltic pump. The peristaltic pump can transport the liquid by pressing and releasing the peristaltic tubes (not shown) thereof by turns, and the liquid therein can be isolated within the peristaltic tubes without contact with other elements of the peristaltic pump. Accordingly, the peristaltic pump has advantages of low contaminate rate and continuous fluid delivery. Therefore, it is favorable for the microenvironment-simulated cell culture system 100 of the present application to screen the anticancer drugs without being affected by external substances, and thus the microenvironment-simulated cell culture system 100 of the present application has the potential for clinical application.

Therefore, by the arrangement that the cells are cultured in the three-dimensional space of the cell culture chamber 112 of the cell culture chip 110, a three-dimensional growing tumor model can be created in the microenvironment-simulated cell culture system 100 of the present disclosure in a short period of time. Further, by the arrangement that the cell culture chamber 112 is a long-stripped slot and the first side portion 1121 and the second side portion 1122 are respectively disposed on the two ends of the cell culture chamber 112, the area where the substances can be exchanged between the cell culture chamber 112 and the external space is restricted to the first side portion 1121. At the same time, the fluid circulation system established by the fluid driving member 130 communicates the first side portion 1121 and flows cyclically, so that the oxygen, nutrients and other substances in the cell culture chamber 112 can be exchanged at the first side portion 1121, and a molecular gradient that gradually decreases along a direction from the molecular gradient to the second side portion 1122 can be established in the cell culture chamber 112. Thus, it is favorable for more accurately simulating the conditions of oxygen, nutrients and immune cells in tumor clinically, so that the anticancer drugs for different types of cancer can be screened, and the in vivo process of immune cells fighting to the tumors can be simulated. Accordingly, the time required for the conventional experiments can be greatly shortened, and the tests thereof have high reproducibility and potential for clinical application.

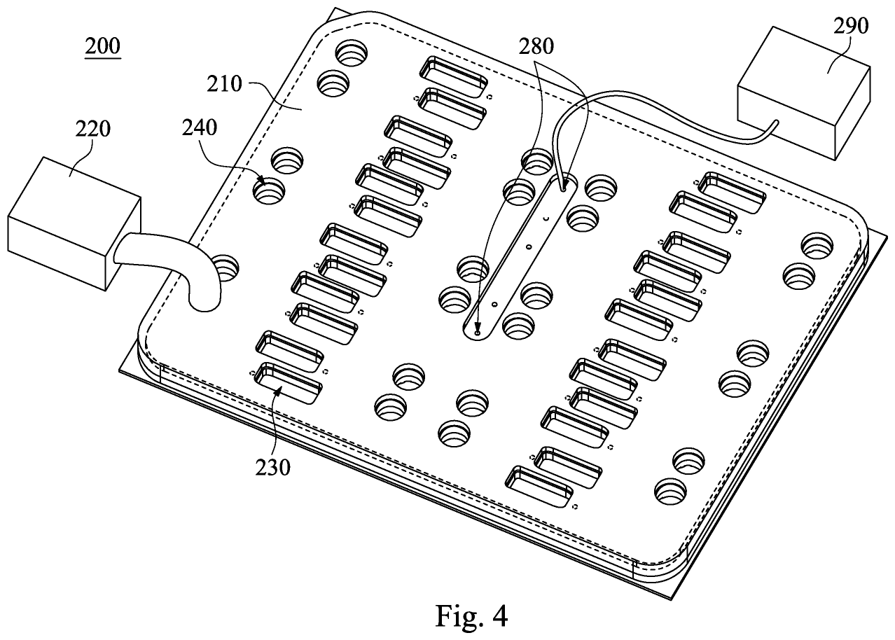

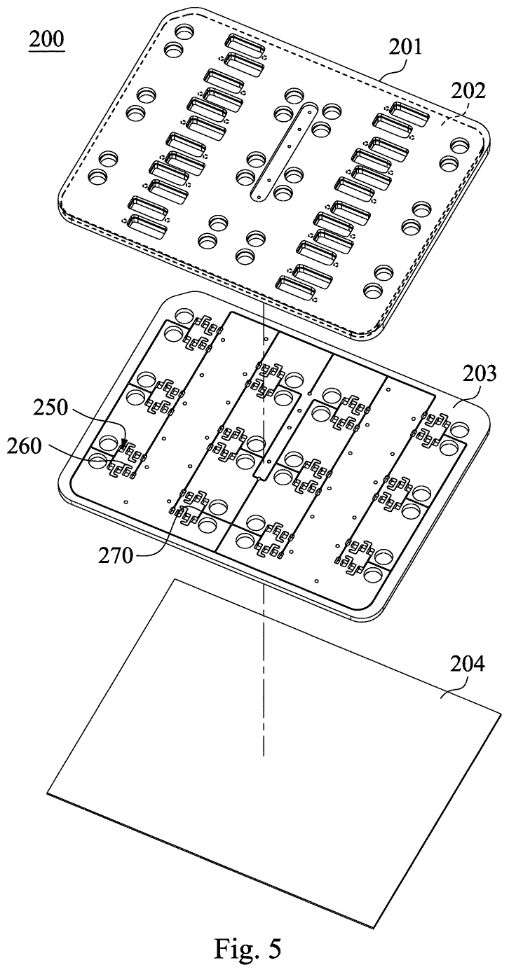

Reference is made to FIG. 4 and FIG. 5, wherein FIG. 4 is a schematic view of a microenvironment-simulated cell culture system 200 according to another embodiment of the present disclosure, and FIG. 5 is an exploded view of the cell culture chip 210 of FIG. 4. The microenvironment-simulated cell culture system 200 includes the cell culture chip 210, a fluid storage device 220 and an air pump 290.

As shown in FIG. 4 and FIG. 5, the cell culture chip 210 includes at least one cell culture chamber 230, at least one fluid storing well 240, at least three membrane pump units 250, at least one air transporting channel 260, at least one valve 270 and at least two air transporting pores 280. The fluid storage device 220 is connected to the at least one fluid storing well 240, and the fluid storage device 220 is for supplying a cell culture medium to the at least one fluid storing well 240. The air pump 290 is connected to the at least two air transporting pores 280.

The at least one cell culture chamber 230 includes a first side portion and a second side portion, and the first side portion and the second side portion are respectively disposed on two ends of the at least one cell culture chamber 230 along a long axis of the cell culture chip. In detail, the at least one cell culture chamber 230 is substantially a long-stripped slot, two long sides of the at least one cell culture chamber 230 are parallel to the long axis of the cell culture chip 210, a length ratio of a short side of the at least one cell culture chamber 230 to one of the long sides of the at least one cell culture chamber 230 is 1:1 to 1:4, and a length of the one of the long sides of the at least one cell culture chamber 230 is 1.3 cm to 2.0 cm. Further, the first side portion, the second side portion, the long sides and the short sides are the same as those of the microenvironment-simulated cell culture system 100, and the details thereof are not be described herein.

The at least one fluid storing well 240 is connected to the at least one cell culture chamber 230. The at least three membrane pump units 250 are connected to the at least one fluid storing well 220, and the at least three membrane pump units 250 are arranged in sequence. The at least one air transporting channel 260 is connected among the at least three membrane pump units 250, wherein the at least one air transporting channel 260 is substantially s-shaped, and a width of the air transporting channel 260 is 0.06 mm to 0.12 mm. The at least one valve 270 is connected between the air transporting channel 260 and the air pump 290. One of the at least two air transporting pores 280 is connected to the at least three membrane pump units 250, and the other one of the at least two air transporting pores 280 is connected to the at least one valve 270.

In detail, in the microenvironment-simulated cell culture system 200, the cell culture chamber 230 is more than one, and the plural cell culture chambers 230 are separately arranged in the cell culture chip 210. One cell culture chamber 230 corresponds to one fluid storing well 240, three membrane pump units 250, one air transporting channel 260 and one valve 270. One of the air transporting pores 280 is for controlling the valve 270 to open or close, and the other one of the air transporting pores 280 is for controlling the at least three membrane pump units 250 to work. The valve 270 can be opened by the control of the air pump 290 in need, and then the at least three membrane pump units 250 can work in sequence. At this time, because the air transporting channel 260 is substantially s-shaped, the gas flow driven by the air pump 290 in the air transporting channel 260 can be reduced gradually, so that the gas flow among the at least three membrane pump units 250 are time-delayed. Therefore, a peristaltic pushing effect of the cell culture medium from the fluid storing well 240 to the cell culture chamber 230 can be achieved, and a cultured condition of the cell in the cell culture chamber 230 can be controlled.

Further, as shown in FIG. 4 and FIG. 5, the cell culture chip 210 is composed of a first base plate 201, an upper base plate 202, a middle base plate 203 and a lower base plate 204. The first base plate 201, the upper base plate 202, the middle base plate 203 and the lower base plate 204 are stacked in sequence. Further, the first base plate 201, the upper base plate 202, the middle base plate 203 and the lower base plate 204 can be made of an impermeable material. The impermeable material can be polydimethylsiloxane, poly (methyl methacrylate), polyethylene terephthalate, acrylic, polycarbonate, polystyrene, silicone rubber or glass, and the present disclosure is not limited thereto. Further, although the figure is not shown, the first base plate 201 is sealed by an airtight tape to prevent from air leaking, and the thickness of the airtight tape is more than 0.25 mm. Furthermore, a height of the cell culture chamber 112 perpendicular to the long axis of the mainbody 111 can be 180 μm to 220 μm, and a length of the short side of the cell culture chamber 112 can be 0.5 cm to 1.0 cm so as to achieve a direct optical visualization across the full depth of the construct.

Further, although the figure is not shown, a force can be applied to the cell culture chip 210 from the top thereof before observation, so that a predetermined flatness of the cell culture chip 210 can be maintained during observation, allowing the culture area to be imaged under a single focal plane, but the present disclosure is not limited thereto.

### [Microenvironment-Simulated Cell Culturing Method of the Present Disclosure]

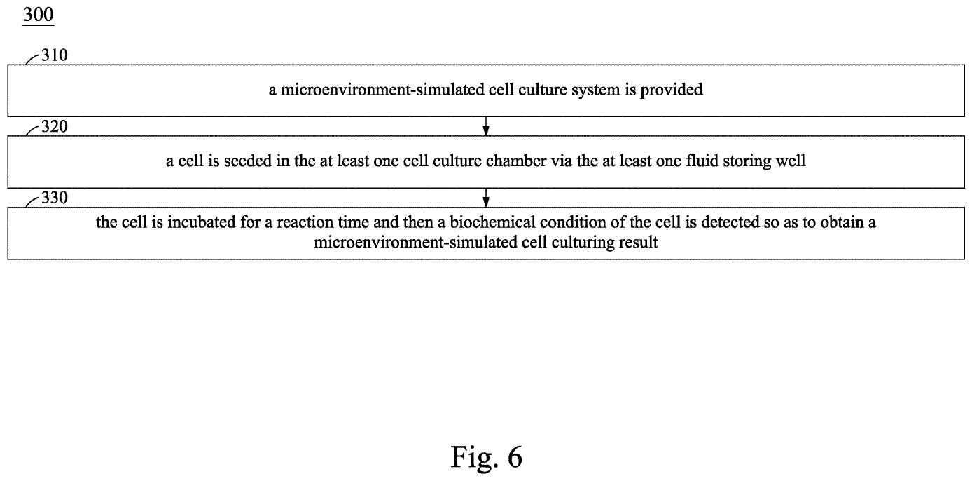

Reference is made to FIG. 6, which is a flow chart of a microenvironment-simulated cell culturing method 300 according to another embodiment of the present disclosure. The microenvironment-simulated cell culturing method 300 includes Step 310, Step 320 and Step 330.

In Step 310, a microenvironment-simulated cell culture system is provided. In detail, the microenvironment-simulated cell culture system can be the microenvironment-simulated cell culture system 200 of FIG. 4, and the details will not be described herein again.

In Step 320, a cell is seeded in the at least one cell culture chamber via the at least one fluid storing well, wherein the air pump drives the at least three membrane pump units in sequence to transport the cell culture medium including the cell in the at least one fluid storing well to the at least one cell culture chamber, and a number of the cell seeded in the at least one cell culture chamber is 0.25×10⁶ to 2.5×10⁷. In detail, the density of the cell seeded in the cell culture chamber will affect the oxygenation profiles in the cell culture chamber, so the number of the cell seeded in the cell culture chamber can be adjusted based on the volume of the cell culture chamber, and the cell can be introduced into the cell culture chamber at varying densities so as to establish the oxygen gradient in different areas of the cell culture chamber. Further, the cell can include a mammalian cell and at least one cancer cell, wherein the at least one cancer cell can be derived from a live tumor tissue. In detail, the live tumor tissue can include the tumor tissues originating from animal models, such as mouse tumors (for example, the breast cancer, the colorectal cancer, and the pancreatic cancer), as well as human solid tumors, including the breast cancer, the pancreatic cancer, the liver cancer, etc. Further, the mammalian cell can include a fibroblast and an immune cell, but the present disclosure is not limited thereto. Hence, the microenvironment-simulated cell culturing method 300 of the present disclosure enables the formation and analysis of the tissues composed of single or multiple cellular components under controlled microenvironmental conditions, allowing investigation of cellular interactions, microenvironmental responses, and spatially distributed biological behaviors.

In Step 330, the cell is incubated for a reaction time and then a biochemical condition of the cell is detected so as to obtain a microenvironment-simulated cell culturing result. In detail, the reaction time can be 24 hours to 72 hours based on the type of cell, but the present disclosure is not limited thereto. Further, the cell can be observed directly from the cell culture chamber to analyze the biochemical condition thereof and also can be harvested and retrieved from the cell culture chamber for downstream analysis of following experimental process, but the present disclosure is not limited thereto. The cells can be analyzed using the flow cytometry to quantify cellular composition, including tumor cells and associated stromal or immune cell populations. This enables evaluation of cell population changes, phenotypic shifts, and treatment-associated cellular dynamics. Accordingly, the microenvironment-simulated cell culturing method 300 of the present disclosure allows the post-culture recovery of biological samples for integrated downstream analyses, including genomic, molecular, and cytometric characterization, thereby providing comprehensive assessment of tumor biology and microenvironmental responses.

Further, a positive pressure of the air pump is 3 to 12 psi, and a negative pressure of the air pump is −3 to −12 psi. In detail, when the positive pressure of the gas pump is 3 to 12 psi and the negative pressure is −3 to −12 psi, a molecular gradient can be formed in the cell culture chamber, and when the positive pressure and the negative pressure of the air pump are outside the aforementioned ranges, the molecular gradient may collapses. Further, the valve only opens when the negative pressure is less than −8 psi, allowing the cell culture medium to circulate; and when the positive pressure is between 3 and 12 psi and the negative pressure is between −3 and −12 psi, the air pump has sufficient torque to drive the cell culture medium through circulation.

Further, a flow rate of the cell culture medium driven by the air pump is 30 μL/min to 60 μL/min. In detail, the dynamic conditions can enhance the nutrient and oxygen transports, remove the metabolic wastes and maintain the physiologically relevant gradients, thereby supporting preservation of the cell heterogeneity and native states. Further, compared to the static culture, the dynamic circulation better preserves characteristics of the original tumor, including cellular composition, microenvironmental features, and molecular profiles, and the constructed tumor tissue established under the dynamical condition exhibits higher similarity to the corresponding original tumor tissue, whereas the static culture shows greater deviation over time. Thus, by the flow rate of the cell culture medium driven by the fluid driving member is set to 30 μL/min to 60 μL/min, a stable oxygen diffusion gradient can be established across the cell culture chamber under dynamic circulation conditions, resulting in spatially distinguishable regional oxygen levels. Furthermore, when the positive pressure being 3 to 12 psi and the negative pressure being −3 to −12 psi driven by the air pump are used to achieve a flow rate of the cell culture medium between 3.5 L/min and 10.5 L/min, and the air transporting channel has a size of 0.12 mm×0.06 mm and a length between 8 mm and 10 mm, the membrane pump units exhibit a sequential peristaltic effect.

Therefore, by the pneumatically driven fluid control method of the microenvironment-simulated cell culturing method 300, the plurality of the cell culture chambers can be synchronously or correlatedly controlled by a single control source, so that the fluid transport delays and inter-unit differences can be reduced. Further, under specific operating conditions, a stable concentration distribution can be formed within the cell culture chamber, and thus the fluid correlations can be maintained between different cell culture chambers, thereby improving experimental reproducibility and analytical reliability of the microenvironment-simulated cell culturing method 300.

### Examples

The simulating effects of the actual tumor microenvironment of the microenvironment-simulated cell culture system of the present disclosure will be further exemplified by performing the cell cultivation with the microenvironment-simulated cell culture system of the present disclosure, and the experiments will be further conducted with different drugs or immune cells. However, the readers should understand that the present disclosure should not be limited to these practical details thereof, that is, in some embodiments, these practical details are used to describe how to implement the materials and methods of the present disclosure and are not necessary.

The following experiments are performed by the microenvironment-simulated cell culture system of the present disclosure. In the experiments, the cell culture chamber of the cell culture chip is equally divided into a normoxia area and an hypoxia area along a direction from the first side portion to the second side portion, wherein the normoxia area is further equally divided into Area 1 and Area 2 along the direction from the first side portion to the second side portion, the hypoxia area is also further equally divided into Area 3 and Area 4 along the direction from the first side portion to the second side portion, and the order of the oxygen concentration is: Area 1>Area 2>Area 3>Area 4. Further, a transition zone between the normoxia area and the hypoxia area is defined by that Area 2 and Area 3 are respectively divided into two regions, and the two adjacent regions of Area 2 and Area 3 are the transition zone.

The following tests are respectively performed by co-culturing 4T1 mouse breast cancer cells (“4T1 cells” hereafter) and the K-BALB fibroblasts (“K-BALB cells” hereafter) in the microenvironment-simulated cell culture systems of Example 1 to Example 3, wherein 4T1 cells belong to a triple-negative breast cancer cell line and are often used as a research model for distant metastasis of breast cancer and a clinical drug screening model, and K-BALB cells is a fibroblast cell line homologous to 4T1 cells. Further, the cell culture medium for culturing 4T1 cells is the 89% high glucose DMEM including 10% fetal bovine serum (FBS) and 1% Penicillin/Streptomycin solution (P/S), and the cell culture medium for culturing K-BALB cells is the 89% high glucose DMEM including 10% bovine calf serum and 1% Penicillin/Streptomycin solution. 4T1 cells and K-BALB cells are co-cultured at 37° C., 5% CO₂ for 24 hours so as to carry out different analysis, and the growth statuses of 4T1 cells and K-BALB cells are observed and the molecular expression in the areas with different oxygen concentrations are analyzed.

Further, in the following tests, a length ratio of a short side to a long side of the cell culture chamber of the microenvironment-simulated cell culture system of Example 1 is 1:4, a length ratio of a short side to a long side of the cell culture chamber of the microenvironment-simulated cell culture system of Example 2 is 1:2, and a length ratio of a short side to a long side of the cell culture chamber of the microenvironment-simulated cell culture system of Example 3 is 1:1. Furthermore, the cell culture chip, the fluid storage device and the air pump of each of the microenvironment-simulated cell culture systems of Example 1 to Example 3 are the same as that of the microenvironment-simulated cell culture system 100 of FIG. 1, so that the details of the same structures or the arrangements are shown in the aforementioned paragraphs and will not be described herein again.

### I. 4T1 Cells and K-BALB Cells are Cultured in the Microenvironment-Simulated Cell Culture System of the Present Disclosure

In the present test, the cell suspensions including 4T1 cells and K-BALB cells are respectively transported into the cell culture chamber through the sample loading well of each of the microenvironment-simulated cell culture systems of Example 1 to Example 3 first, and then the sample loading well is closed so as to facilitate the attachment and growth of 4T1 cells and K-BALB cells in the cell culture chamber. At the same time, the air pump will drive the cell culture medium in the fluid storage device transporting through one of the fluid delivery ports to the cell culture chamber and then moving out of the cell culture chamber through the other one of the fluid delivery ports, and the cell culture medium will continue to flow and circulate along the aforementioned path so as to establish a dynamic fluid circulation system in the cell culture chip. Next, the microenvironment-simulated cell culture systems of Example 1 to Example 3 will be maintained under 37° C., 5% CO₂ for 24 hours so as to facilitate the three-dimensional growth of 4T1 cells and K-BALB cells and then process the subsequent analysis.

Furthermore, it is noted that if the methods and details of the following experiments are known in the art, they will not be described in detail.

### II. Analysis of the Oxygen Concentration Gradient in the Cell Culture Chip

In the present test, the expressions of the hypoxia-inducible factor 1-alpha (“HIF 1-α” hereafter) in different areas of the microenvironment-simulated cell culture systems of Example 1 to Example 3 are analyzed. In detail, HIF 1-α is a transcription factor in the cellular environment that is activated in the conditions of oxygen reduction or hypoxia. If the expression of HIF 1-α is higher, the oxygen concentration in the area is lower. Thus, the protein expressions of HIF 1-α of the cells in different areas are further analyzed by Western blotting method, and the cells cultured in the microenvironment-simulated cell culture systems of Example 1 to Example 3 are stained with the fluorescent dye of Invitrogen™ Image-iT™ Red Hypoxia Reagent so as to assess whether the oxygen concentration gradient is established in the cell culture chamber or not.

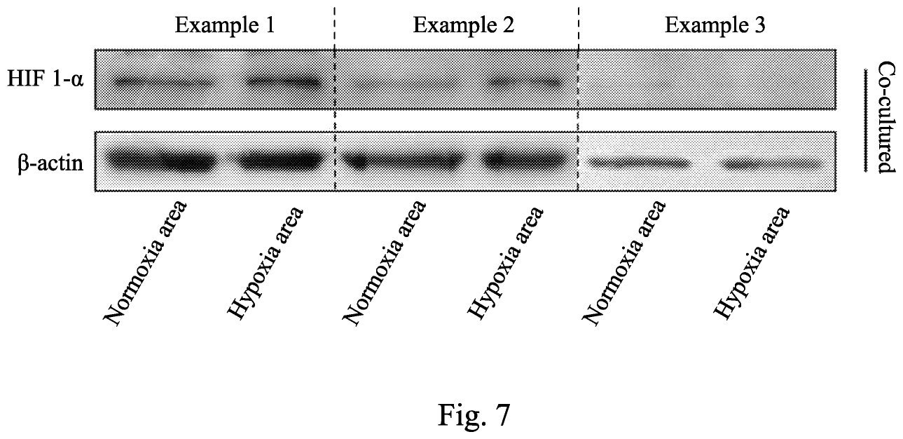

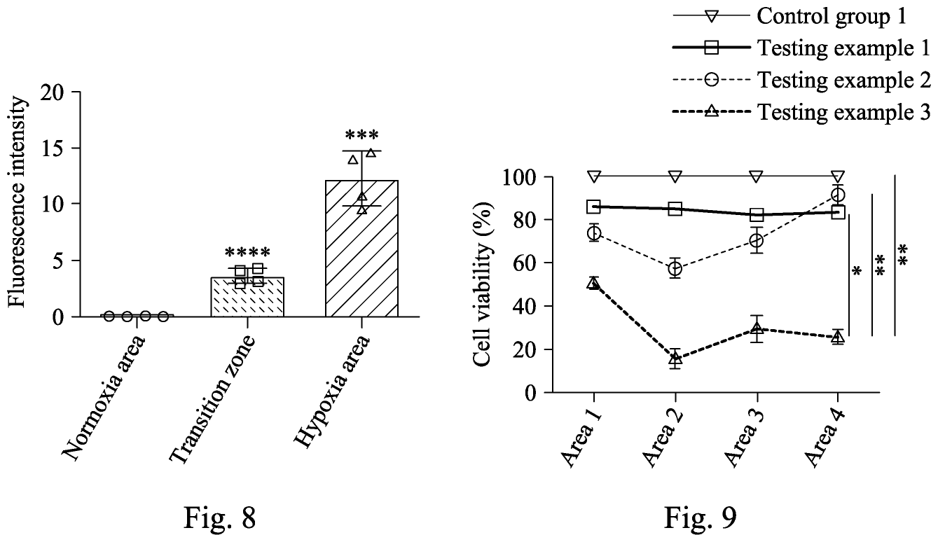

Reference is made to FIG. 7 and FIG. 8. FIG. 7 shows the results of Western blotting analysis of the co-cultivation of 4T1 cells and K-BALB cells for 24 hours by the microenvironment-simulated cell culture systems of Example 1 to Example 3 (hereafter referred to as “Example 1”, “Example 2” and “Example 3”). FIG. 8 shows the analysis results of hypoxia signal quantification in different areas of the cell culture chamber of the microenvironment simulated cell culture system of Example 2. As shown in FIG. 7, the expressions of HIF 1-α in the hypoxia areas of Example 1 and Example 2 increase, and the largest difference in expression is shown in Example 2. Further, in FIG. 8, the definition of the fluorescence intensity is the relative fluorescence intensity calculated based on the fluorescence intensity in the normoxia area as 1. As shown in FIG. 8, the hypoxia signals gradually increase along the direction from the normoxia area, the transition zone to the hypoxia area and have a significant increase in the hypoxia area.

As shown in the aforementioned results, the oxygen concentration gradient can be established in the cell culture chamber of the microenvironment-simulated cell culture system of the present disclosure, and it has the potential for use in relevant clinical trials.

### III. Analysis of the Effects of the Gradient of Small Molecule Drugs on Cytotoxicity

In the present test, 4T1 cells and K-BALB cells are co-cultured in the microenvironment-simulated cell culture system of Example 2 so as to observe the effects of the small molecule drugs on 4T1 cells and K-BALB cells after being diffused from the cell culture medium to the cell culture chamber. In the present test, Control group 1 is performed with the cell culture medium without any drug, Testing example 1 is performed with the cell culture medium including Gemcitabine, Testing example 2 is performed with the cell culture medium including Galunisertib (an inhibitor of TGF-β1), and Testing example 3 is performed with the cell culture medium including Gemcitabine and Galunisertib. In detail, Gemcitabine is a synthetic cytosine derivative clinically used for the cancer chemotherapy, and it has the advantages of a strong radiosensitization effect and less toxic side effects. Further, Galunisertib, which is an inhibitor of the cytokine TGF-31 closely related to the biochemical pathway of drug resistance, is further used in the present test, and Gemcitabine and Galunisertib are combined to use so as to observe the cells after being treated with the aforementioned combination.

In the present test, the concentration of Gemcitabine in the cell culture medium is 100 μM, and the concentration of Galunisertib in the cell culture medium is 100 μM. At the same time, the cell viabilities of 4T1 cells and K-BALB cells in different areas are further analyzed, and 4T1 cells and K-BALB cells are stained with the propidium iodide so as to observe the effects of different drug combinations on 4T1 cells and K-BALB cells cultured in the cell culture chamber of the cell culture chip of the microenvironment-simulated cell culture system of the present disclosure.

Reference is made to FIG. 9, which shows the analysis results of cell viability in different areas of the cell culture chamber after culturing for 24 hours with different drug combinations. As shown in FIG. 9, the cell viabilities of Area 1 to Area 4 of Testing example 1 are not significantly different there between, but the cell viabilities of Area 1 to Area 4 of Testing example 3 are significantly lower than that of other testing examples. Further, in Testing example 3, the cell viability of Area 4 is significantly lower than that of Area 1. Accordingly, the concentration gradient of drugs can be established in the cell culture chamber of the cell culture chip of the microenvironment-simulated cell culture system of the present disclosure, and can be used to simulate the growth state of tumor cells during drug administration.

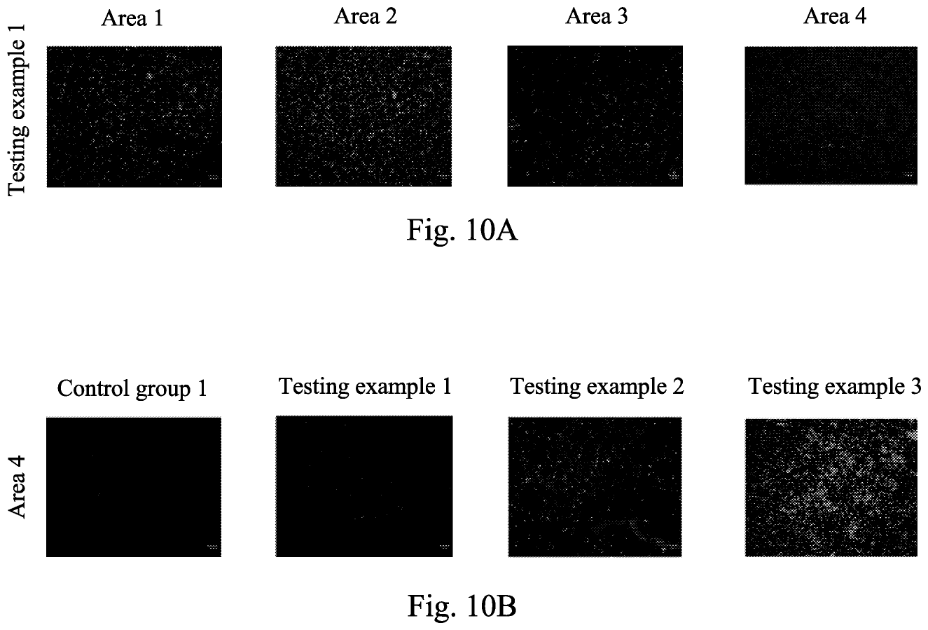

Reference is further made to FIG. 10A and FIG. 10B. FIG. 10A shows the staining results of the propidium iodide in different areas of the cell culture chamber of Testing example 1, and FIG. 10B shows the staining results of the propidium iodide in Area 4 of the cell culture chambers of Testing example 1 to Testing example 4. As shown in FIG. 10A, signals of the propidium iodide in Area 1 and Area 2 of Testing example 1 are significantly higher than that of Area 3 and Area 4. Accordingly, it is shown that the concentrations of Gemcitabine in Area 1 and Area 2 are higher, so that the apoptosis of 4T1 cells and K-BALB cells can be induced, and the inhibition of apoptosis increase in the hypoxia area (that is, Area 3 and Area 4). Further, as shown in FIG. 10B, when lonely comparing the intensities of the propidium iodide signal in Areas 4, which is with the lowest oxygen concentration, of Testing example 1 to Testing example 3, the intensity of the propidium iodide signal of Testing example 3, which has a better drug treating performance in FIG. 10A, is significantly higher than that of other examples. Accordingly, it is shown that different drug combinations can still have different effects on the cells in the areas with different oxygen concentrations in the cell culture chamber, and the results of the aforementioned tests are highly similar to the performance of the tumor microenvironment in clinical. Therefore, the microenvironment-simulated cell culture system of the present disclosure has an excellent ability to be used to study the interaction between drugs and oxygen gradients in the tumor microenvironment, and has excellent potential for clinical application.

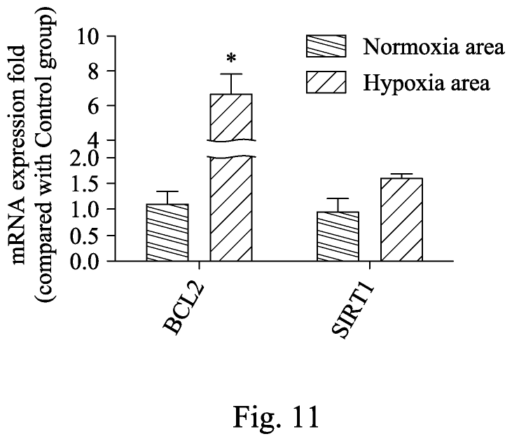

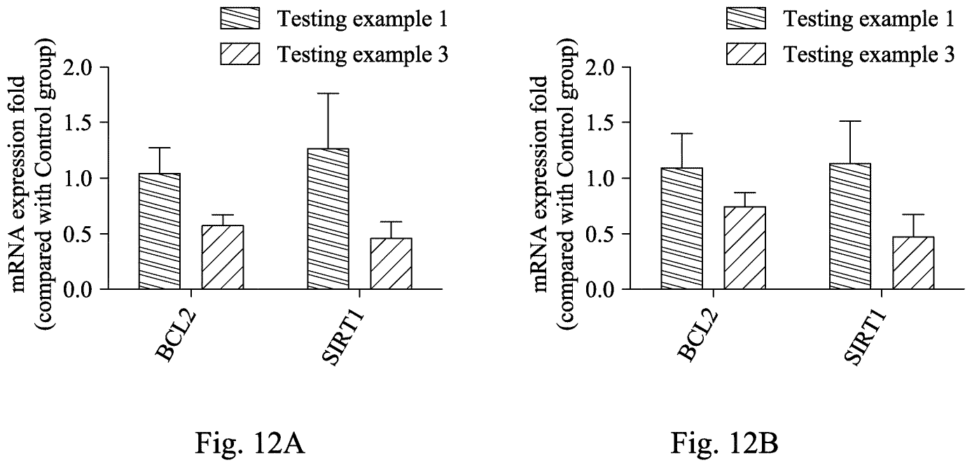

Reference is further made to FIG. 11, FIG. 12A and FIG. 12B simultaneously. FIG. 11 shows the quantification results of mRNA expression in the normoxia area and the hypoxia area of the cell culture chamber of Testing example 1, FIG. 12A shows the quantification results of mRNA expression in the normoxia areas of the cell culture chambers of Testing example 1 and Testing example 3, and FIG. 12B shows the quantification results of mRNA expression in the hypoxia areas of the cell culture chambers of Testing example 1 and Testing example 3. In detail, in the present test, qPCR is simultaneously used to measure the expression of mRNA of proteins related to apoptosis and drug resistance in the areas with different oxygen concentrations in the cell culture chamber of Testing example 1 and Testing example 3, wherein BCL2 is an apoptosis-regulating protein that can regulate cell death by inhibiting or inducing apoptosis, while SIRT1 can inactivate the drug by removing the acetyl group of the target protein.

As shown in FIG. 11, both of the mRNA expressions of BCL2 and SIRT1 in the hypoxia area of Testing example 1 are higher than that of the normoxia area. However, as shown in FIG. 12A and FIG. 12B, after treating with Galunisertib adjunctively, the mRNA expressions of BCL2 and SIRT1 significantly decrease in the normoxia area and the hypoxia area. Accordingly, it is shown that the microenvironment-simulated cell culture system of the present disclosure can indeed be used to simulate the effects of small molecule drugs on tumors, and it is consistent with the current clinical research results and has application potential in the relevant market.

### IV. Analysis of the Expressions of Immune or Inflammatory Response-Related Proteins

In the present test, 4T1 cells and K-BALB cells are co-cultured in the microenvironment-simulated cell culture system of Example 2 so as to observe the mRNA expression of the proteins related to the immunity or inflammatory responses in different areas of the cell culture chamber. In the present test, Control group 2 is performed by culturing K-BALB cells using the cell culture medium without any drug; Testing example 4 is performed by co-culturing 4T1 cells and K-BALB cells using the cell culture medium without any drug, and then K-BALB cells are separated by magnetic beads for analysis; Testing example 5 is performed by co-culturing 4T1 cells and K-BALB cells using the cell culture medium including 50 μM Galunisertib, and then K-BALB cells are separated by magnetic beads for analysis; and Testing example 6 is performed by co-culturing 4T1 cells and K-BALB cells using the cell culture medium including 50 μM AZD-1480 (JAK1/2 inhibitor), and then K-BALB cells are separated by magnetic beads for analysis. Further, Control group 3 is performed by co-culturing 4T1 cells and K-BALB cells using the cell culture medium without any drug, and then 4T1 cells are separated by magnetic beads for analysis; Testing example 7 is performed by co-culturing 4T1 cells and K-BALB cells using the cell culture medium including 50 μM Galunisertib, and then 4T1 cells are separated by magnetic beads for analysis; and Testing example 8 is performed by co-culturing 4T1 cells and K-BALB cells using the cell culture medium including 50 μM AZD-1480, and then 4T1 cells are separated by magnetic beads for analysis. After culturing for 24 hours, the expressions of the immune or the inflammatory response-related proteins in the normoxia area and the hypoxia area of the cell culture chamber of each of Control group 2, Control group 3 and Testing example 4 to Testing example 8 are further measured.

In particular, the leukemia inhibitory factor (“LIF” hereafter) is a cytokine belonging to the interleukin 6 class cytokine and can affect the cell by inhibiting the differentiation thereof; the transforming growth factor-31 (“TGF-31” hereafter) can regulate the growth, the proliferation and the differentiation of the cell as well as regulate whether the apoptosis happens or not; the programmed cell death ligand 1 (“PD-L1” hereafter) is an important regulatory protein for the initiation of immune function in vivo; the collagen I (“col-I” hereafter) is associated with the differentiation of tumor or the inflammatory response; and the interleukin-1 (“IL-1” hereafter) plays an important role in controlling of immune and inflammatory responses.

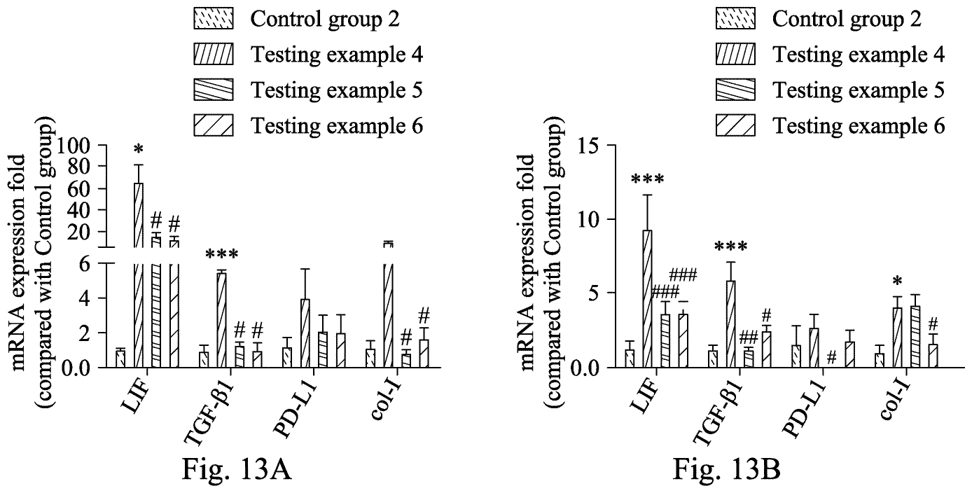

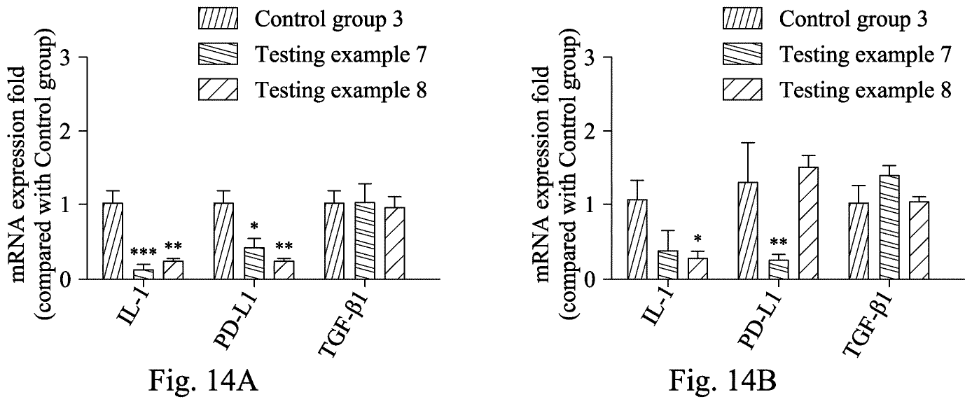

Reference is made to FIG. 13A, FIG. 13B, FIG. 14A and FIG. 14B. FIG. 13A shows the quantification results of mRNA expression of the K-BALB cells in the normoxia areas of the cell culture chambers of Control group 2 and Testing example 4 to Testing example 6, FIG. 13B shows the quantification results of mRNA expression of the K-BALB cells in the hypoxia areas of the cell culture chambers of Control group 2 and Testing example 4 to Testing example 6, FIG. 14A shows the quantification results of mRNA expression of 4T1 cells in the normoxia areas of the cell culture chambers of Control group 3, Testing example 7 and Testing example 8, and FIG. 14B shows the quantification results of mRNA expression of 4T1 cells in the hypoxia areas of the cell culture chambers of Control group 3, Testing example 7 and Testing example 8. In FIG. 13A, FIG. 13B, FIG. 14A and FIG. 14B, “*” represents that the statistical data is obtained by being compared with the data of Control group 2 or Control group 3, and “#” represents that the statistical data is obtained by being compared with the data of Testing example 4.

In cells are cultured in the detail, when K-BALB microenvironment-simulated cell culture system of Example 2 alone, some of the signals of fibrosis and inflammation will express in the hypoxia area of the cell culture chamber. Further, when 4T1 cells are cultured in the microenvironment-simulated cell culture system of Example 2 alone, the signals of inflammation will express in the hypoxia area of the cell culture chamber. However, as shown in FIG. 13A and FIG. 13B, after separating K-BALB cells of Testing example 4 to Testing example 6 by the magnetic beads and then analyzing by qPCR, all of the mRNA expressions of LIF, TGF-31, PD-L1 and col-I of K-BALB cells in the normoxia area and the hypoxia area of the cell culture chamber of Testing example 4 significantly increase compared with that of Control group 2, and the mRNA expressions thereof decrease along with the application of Galunisertib or AZD-1480 and have different amounts correspondingly. Further, as shown in FIG. 14A and FIG. 14B, after separating 4T1 cells of Testing example 7 and Testing example 8 by the magnetic beads and then analyzing by qPCR, the mRNA expressions of IL-1 and PD-L1 in the normoxia area and the hypoxia area of the cell culture chamber of Testing example 7 and Testing example 8 significantly decrease compared with that of Control group 3, but both of the expressions of TGF-31 of Testing example 7 and Testing example 8 are comparable to that of Control group 3. Accordingly, it is shown that the cells in the normoxia area and the hypoxia area of the cell culture chamber of the microenvironment-simulated cell culture system of the present disclosure can have different performances of the immune response or the inflammatory response, and the microenvironment-simulated cell culture system of the present disclosure can be used to simulate the effect of drugs on tumors and has application potential in the relevant market.

### V. Analysis of the Content of T Cells in the Cell Culture Chip and the Expression Thereof

In the present test, 4T1 cells, K-BALB cells and T cells expressing CD3 are co-cultured in the microenvironment-simulated cell culture system of Example 2 so as to analyze the survival state of T cells in the microenvironment-simulated cell culture system of the present disclosure, and then the feasibility immunotherapy research of the microenvironment-simulated cell culture system of the present disclosure is assessed. In the present test, T cells are transferred to the cell culture chamber along with the cell culture medium driven by the air pump so as to simulate the state that T cells enter the tumor from the circulatory system during the growth of tumor in the real state. Then, after culturing for 24 hours, the expressions of the receptors associated with T-cell depletion, namely T-cell immunoglobulin domain and mucin domain-3 (“Tim-3” hereafter), the cytotoxic T lymphocyte associated antigen-4 (“CTLA-4” hereafter) and the programmed cell death protein-1 (“PD-1” hereafter), are analyzed, and the expressions of apoptosis-related protein, Caspase 3/7, in different areas of the cell culture chamber are analyzed, simultaneously.

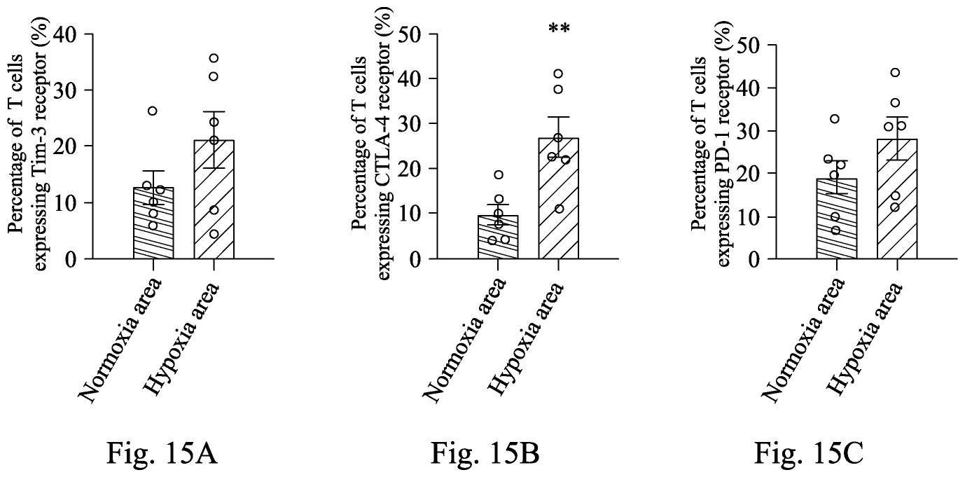

Reference is made to FIG. 15A, FIG. 15B and FIG. 15C. FIG. 15A shows the analysis results of the percentage of T cells expressing Tim-3 receptor in the normoxia area and the hypoxia area of the cell culture chamber, FIG. 15B shows the analysis results of the percentage of T cells expressing CTLA-4 receptor in the normoxia area and the hypoxia area of the cell culture chamber, and FIG. 15C shows the analysis results of the percentage of T cells expressing PD-1 receptor in the normoxia area and the hypoxia area of the cell culture chamber. In particular, T cells are transferred to the cell culture chamber along with the cell culture medium driven by the air pump, and as shown in the results of the pre-analysis of the infiltration of T cells, the amount of T cells in the normoxia area is significantly higher than that in the hypoxia area. Further, as shown in FIG. 15A to FIG. 15C, the proportions of T cells expressing the receptors of Tim-3, CTLA-4 and PD-1 to total cells in the hypoxia area are significantly increased. Accordingly, it is shown that the immunotherapy resistance in the hypoxia area can be observed in the microenvironment-simulated cell culture system of the present disclosure.

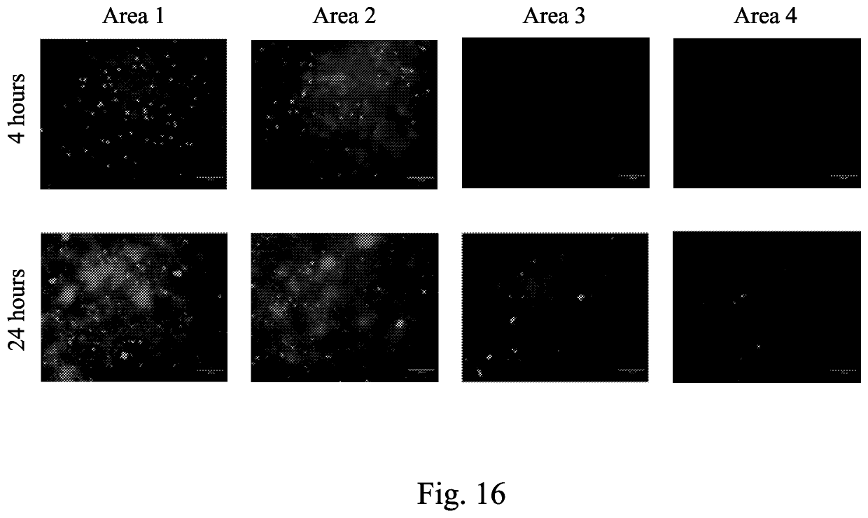

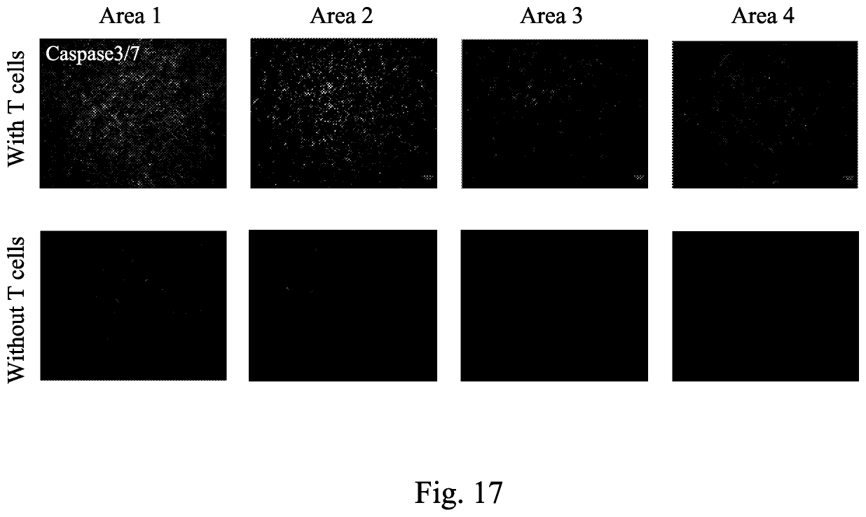

Reference is further made to FIG. 16 and FIG. 17. FIG. 16 shows the staining results of T cells in different areas of the cell culture chamber after being cultured for 4 hours and 24 hours, and FIG. 17 shows the staining results of apoptosis signaling-related proteins in different areas of the cell culture chamber. As shown in FIG. 16, after culturing for 4 hours and 24 hours, the signals of T cells gradually decrease from Area 1 to Area 4 of the cell culture chamber, and thus it is shown that the gradient of the immune cells in the actual environment can be effectively simulated by the cell culture chip of the microenvironment-simulated cell culture system of the present disclosure at different times. Further, as shown in FIG. 17, in the presence of T cells, the expressions of Caspase 3/7 also gradually decrease from Area 1 to Area 4 of the cell culture chamber, and thus it is shown that the apoptosis of cells is suppressed in the hypoxia area. Furthermore, under the condition without T cells, the expressions of Caspase 3/7 are approximately the same in Area 1 to Area 4 of the cell culture chamber. Accordingly, it is shown that the microenvironment-simulated cell culture system of the present disclosure has the potential to apply to the immunotherapy research of cancer and has excellent clinical application potential.

### VI. Analysis of the Effects of the Immune Checkpoint Inhibitor and the Anticancer Drug Therapy

In the present test, 4T1 cells, K-BALB cells and T cells expressing CD3 are co-cultured in the microenvironment-simulated cell culture system of Example 2 so as to analyze the survival state of 4T1 cells, K-BALB cells and T cells after being treated with the immune checkpoints inhibitor and anticancer drugs, and then the feasibility for use in immunotherapy research of the microenvironment-simulated cell culture system of the present disclosure is assessed.

In the present test, Control group 4 is performed with the cell culture medium without any drug, Testing example 9 is performed by treating the cells in the cell culture chamber with low dose anti-PD-1 antibody at 100 ng/ml, and Testing example 10 is performed by treating the cells in the cell culture chamber with high dose anti-PD-1 antibody at 1000 ng/mL. Further, Control group 5 is performed with the cell culture medium without any drug, Testing example 11 is performed by treating the cells in the cell culture chamber with 1000 ng/ml of anti-PD-1 antibody, Testing example 12 is performed by treating the cells in the cell culture chamber with 50 μM of Galunisertib, and Testing example 13 is performed by treating the cells in the cell culture chamber with 1000 ng/ml of anti-PD-1 antibody as well as 50 μM of Galunisertib.

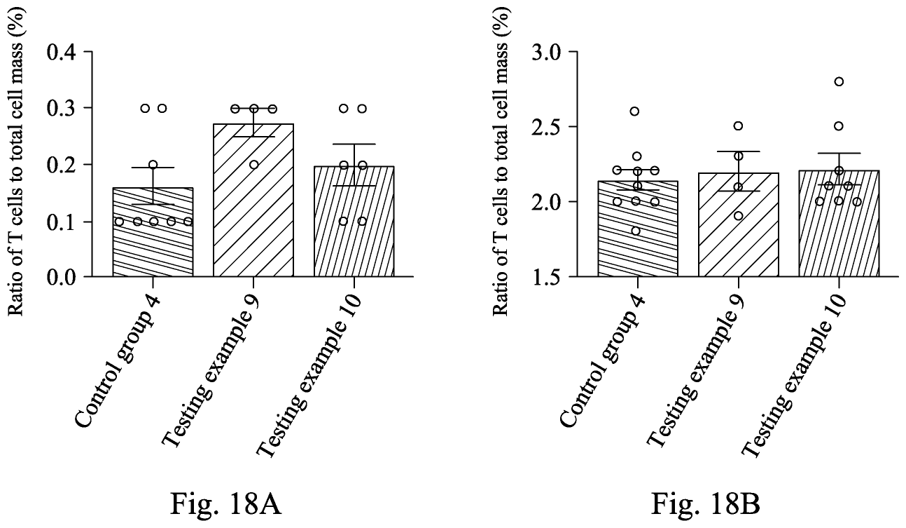

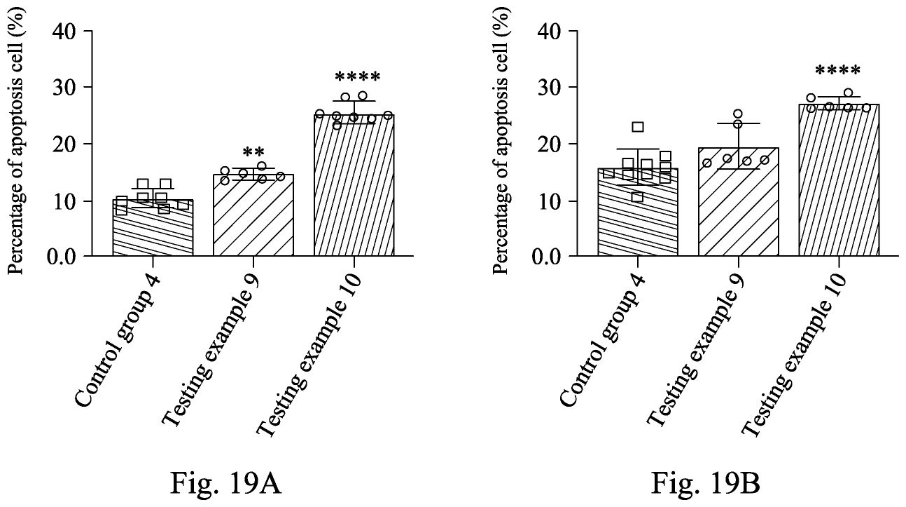

Reference is made to FIG. 18A, FIG. 18B, FIG. 19A and FIG. 19B. FIG. 18A shows the analysis results of the ratio of T cells to the total cell mass in the hypoxia area of the cell culture chamber after being treated with different doses of anti-PD-1 antibody, FIG. 18B shows the analysis results of the ratio of T cells to the total cell mass in the normoxia area of the cell culture chamber after being treated with different doses of anti-PD-1 antibody, FIG. 19A shows the analysis results of the percentage of apoptosis in the hypoxia area of the cell culture chamber after being treated with different doses of anti-PD-1 antibody, and FIG. 19B shows the analysis results of the percentage of apoptosis in the normoxia area of the cell culture chamber after being treated with different doses of anti-PD-1 antibody.

As shown in FIG. 18A and FIG. 18B, after treating by high dose of anti-PD-1 antibody, the infiltration amount of T cells in the hypoxia area of the cell culture chamber of Testing example 9 is higher than that of Testing example 10. Further, in the normoxia area, there is no significant difference between the infiltration amount of T cells in the cell culture chamber of Testing example 9 and that of Control group 4 regardless of whether the low dose of anti-PD-1 antibody or the high dose of anti-PD-1 antibody is administered. However, as shown in FIG. 19A and FIG. 19B, the proportions of apoptosis cells in both of the hypoxia area and the normoxia area are larger than that of Testing example 9 and Testing example 10, wherein the proportions of apoptosis cells in both of the hypoxia area and the normoxia area of Testing example 10 are largest. Accordingly, it is shown that the microenvironment-simulated cell culture system of the present disclosure has the potential to apply to the immunotherapy research of cancer.

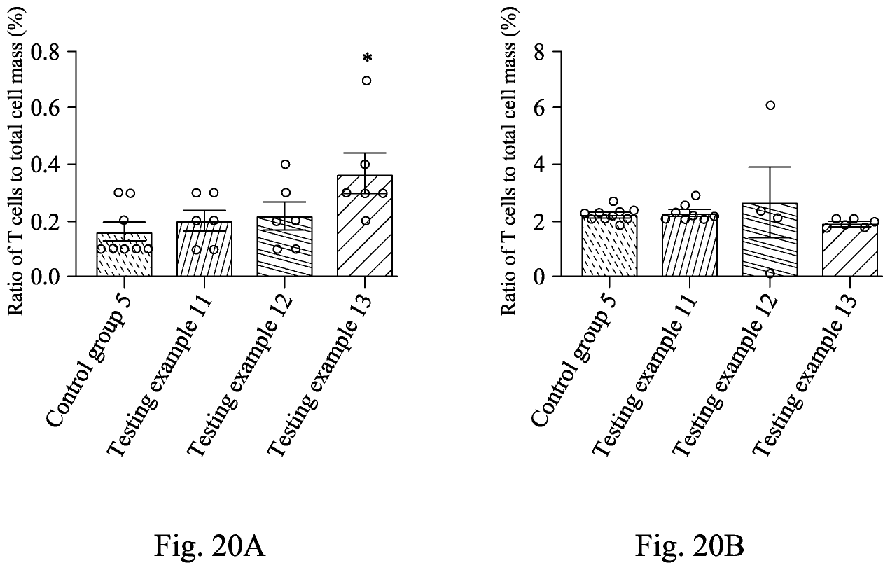

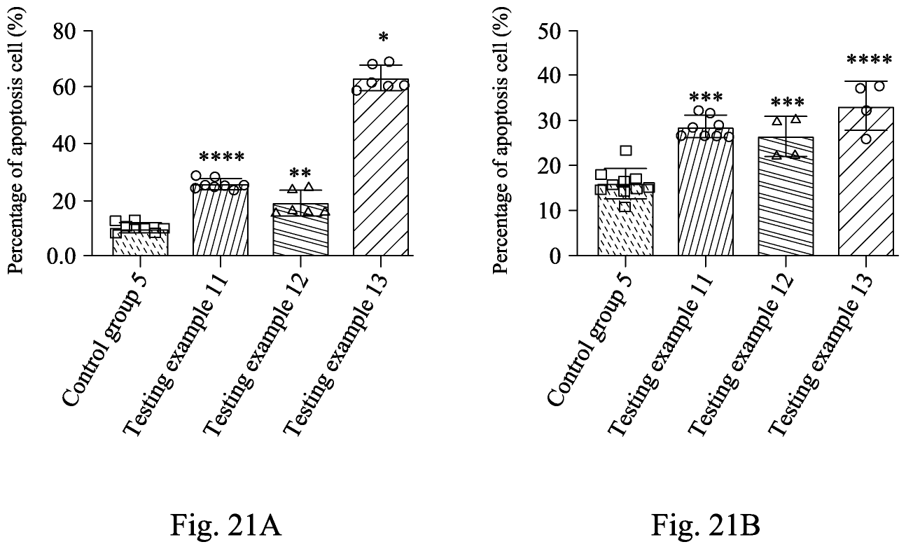

Reference is further made to FIG. 20A, FIG. 20B, FIG. 21A and FIG. 21B. FIG. 20A shows the analysis results of the ratio of T cells to the total cell mass in the hypoxia area of the cell culture chamber after being treated with different drugs, FIG. 20B shows the analysis results of the ratio of T cells to the total cell mass in the normoxia area of the cell culture chamber after being treated with different drugs, FIG. 21A shows the analysis results of the percentage of apoptosis in the hypoxia area of the cell culture chamber after being treated with different drugs, and FIG. 21B shows the analysis results of the percentage of apoptosis in the normoxia area of the cell culture chamber after being treated with different drugs.

As shown in FIG. 20A and FIG. 20B, the infiltration amount of T cells in the hypoxia area of Testing example 13 is the highest, but in the normoxia area, the infiltration amounts of T cells of Testing example 11 to Testing example 13 have no difference compared with that of Control group 5. However, as shown in FIG. 21A and FIG. 21B, all of the proportions of apoptosis 4T1 cells in the hypoxia area and the normoxia area of the cell culture chamber of Testing example 11 to Testing example 13 are larger than that of Control group 5, wherein the proportion of apoptosis cells of Testing example 13 is the highest. Accordingly, it is shown that anti-PD-1 antibody and the Galunisertib have an excellent synergistic inhibitory effect to 4T1 cells.

### VII. Analysis of the Molecule Diffusions in the Cell Culture Chamber of the Microenvironment-Simulated Cell Culture System

In the present test, the microenvironment-simulated cell culture system of Example 4 is used, wherein the microenvironment-simulated cell culture system of Example 4 is the microenvironment-simulated cell culture system 200 of FIG. 4, the length ratio of the short side of the cell culture chamber to the one of the long sides of the cell culture chamber is 1:2, a length of the one of the long sides of the cell culture chamber is 1.3 cm to 2.0 cm, the height of the cell culture chamber perpendicular to the long axis of the cell culture chip is 180 μm to 220 μm, and the length of the short side of the cell culture chamber is 0.5 cm to 1.0 cm. Further, the cells are cultured in the cell culture chamber of the microenvironment-simulated cell culture system of Example 4 according to the microenvironment-simulated cell culturing method 300 of the present disclosure, and the details of the microenvironment-simulated cell culturing method are shown in FIG. 6 and will not be described herein again.

In the experiment, the 4T1 cells are incubated in the cell culture chamber of the microenvironment-simulated cell culture system of Example 4 for 24 hours along with the small molecule fluorescein and the large molecule GFP introduced therein, and then the fluorescence intensities in different areas of the cell culture chamber are quantified. Further, the oxygen distribution is evaluated using the Image-iT™ hypoxia probe, and the fluorescein and the GFP signals are analyzed by using fluorescence microscopy, wherein the excitation/emission wavelengths for detecting the fluorescein is Ex/Em: 488/520 nm, the excitation/emission wavelengths for detecting the GFP is Ex/Em: 488/509 nm, and the excitation/emission wavelengths for detecting the Image-iT™ hypoxia probe is Ex/Em: 488/520 nm. Further, a static condition and a dynamic condition are respectively established, wherein the static condition refers to that the flow rate of the cell culture medium driven by the air pump is 0 μL/min (that is, the air pump is turned off), and the dynamic condition refers to that the flow rate of the cell culture medium driven by the air pump is 30 μL/min to 60 μL/min.

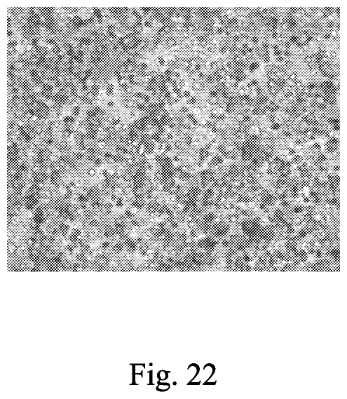

Reference is made to FIG. 22, which shows an image of the 4T1 cells cultured in the cell culture chamber of the microenvironment-simulated cell culture system of Example 4. In detail, after the 4T1 cells are seeded in the cell culture chamber of the microenvironment-simulated cell culture system of Example 4, the 4T1 cells will proliferate and then differentiate to from a constructed tissue. Because the height of the cell culture chamber perpendicular to the long axis of the mainbody in Example is 180 μm to 220 μm, and the thickness of the constructed tissue is approximately 200 μm, enabling direct optical visualization across the full depth of the constructed tissue. Further, due to the arrangement of the height of the cell culture chamber, the spatially resolved area differences can be clearly observed using a standard optical microscope or a fluorescence microscope without the need for sectioning, so that the region-specific cellular behavior, the molecular distribution and the microenvironmental gradients can be monitored directly, and the optical accessibility of the constructed tissue enables real-time observation and continuous recording of dynamic biological processes, such as cellular responses, drug penetrations and microenvironmental changes.

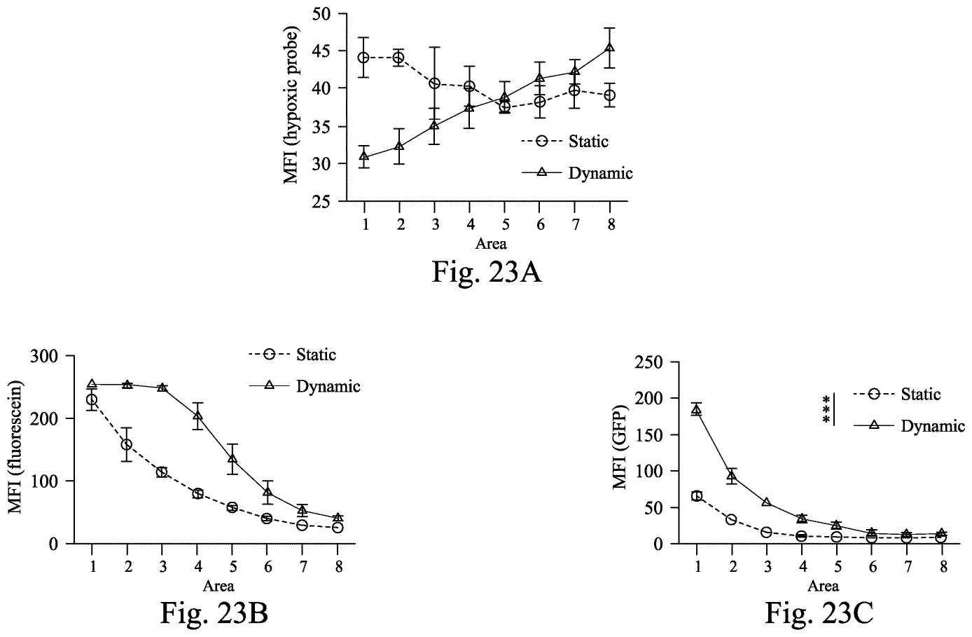

Reference is made to FIG. 23A to FIG. 23C, wherein FIG. 23A shows the diffusion result of oxygen in the cell culture chamber of the microenvironment-simulated cell culture system of Example 4, FIG. 23B shows the diffusion result of micromolecule in the cell culture chamber of the microenvironment-simulated cell culture system of Example 4, and FIG. 23C shows the diffusion result of macromolecule in the cell culture chamber of the microenvironment-simulated cell culture system of Example 4. In FIG. 23A to FIG. 23C, the numbers of the area represent the regions in order from the first side portion to the second side portion of the cell culture chamber, wherein Area 1 is the first side portion and closest to the circulation of the cell culture medium, while Area 8 represents the farthest region away from the circulation of the cell culture medium. The definition of the area shown in the following figures is the same as FIG. 23A to FIG. 23C, and the details will not be described again hereafter.

As shown in FIG. 23A, under the dynamic condition, a stable oxygen diffusion gradient can be established across the cell culture chamber, resulting in spatially distinguishable regional oxygen levels. In contrast, under the static condition, the oxygen distribution fails to form a pronounced gradient. Further, as shown in FIG. 23B and FIG. 23C, the dynamic condition enhances the overall diffusion efficiency and the transport across regions of the micromolecule (fluorescein) and macromolecule (green fluorescent protein, GFP) compared to the static condition. The increased transport enables faster penetration and distribution throughout the constructed tissue.

Further, although the figure is not shown, the cell can be the immune cells. When the immune cells are seeded in the cell culture chamber of the microenvironment-simulated cell culture system of Example 4, the spatially distributed infiltration is established across the cell culture chamber, forming a gradient from the circulation-proximal area to the distal tumor region. The distribution and the penetration of the immune cells vary across regions due to differences in local microenvironmental conditions, including oxygen availability, molecular gradients, and tissue characteristics. As a result of region-specific biological responses, the heterogeneous levels of cytotoxicity in different regions contribute to the extent of cell death being different in the cell culture chamber, the variations in the activity of the immune cells, the sensitivity of the target cells, and the local microenvironmental factors. Accordingly, the microenvironment-simulated cell culture system of the present disclosure enables observation and quantification of regional differences in immune-mediated cellular response and cell death, reflecting physiologically relevant heterogeneous treatment effects.

Therefore, by controlling the circulation and the diffusion, the microenvironment-simulated cell culture and the system microenvironment-simulated cell culturing method of the present disclosure are capable of rapidly generating the desired spatial gradient microenvironment, such as the gradients of oxygen, micromolecule and macromolecular, within a short period of time, thereby mimicking physiologically relevant heterogeneous conditions.

### VIII. Analysis of the Treating Effect of the Chemotherapy Drug on the Cell Cultured in the Cell Culture Chamber of the Microenvironment-Simulated Cell Culture System

In the present test, the 4T1 cells are seeded in the cell culture chamber of the microenvironment-simulated cell culture system of Example 4, and the different concentrations of the doxorubicin are used to treat the 4T1 cells for 24 hours, 48 hours and 72 hours. Then, the 4T1 cells are stained with Caspase 3/7 so as to observe the fluorescence intensity in the 4T1 cells and then measure the apoptosis level thereof. Further, a control group is also provided in the present test, wherein the control group is not treated with any chemotherapy drug, and the data is normalized to the control group.

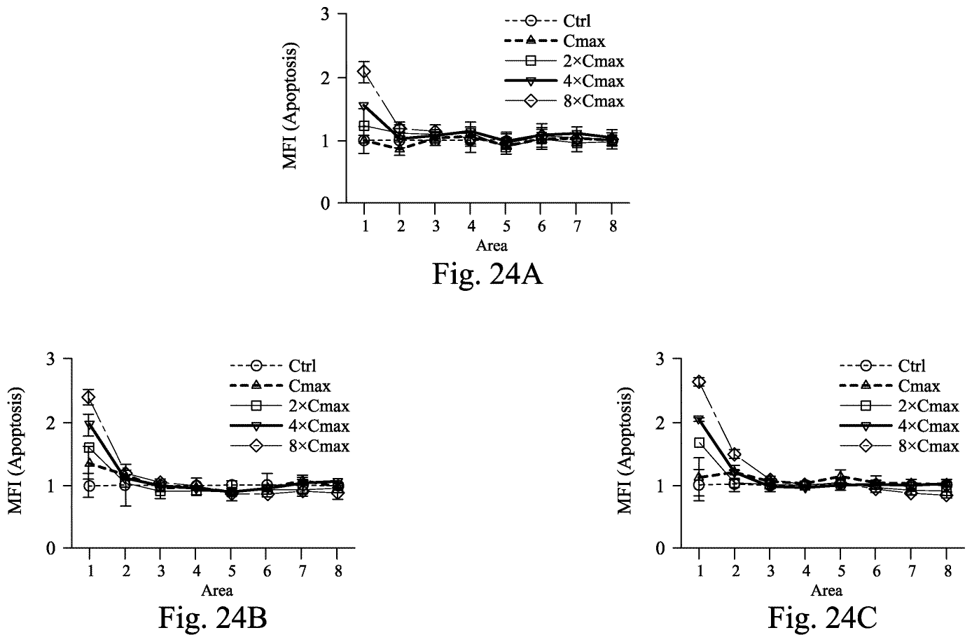

Reference is made to FIG. 24A to FIG. 24C, wherein FIG. 24A shows the analysis results of apoptosis of the 4T1 cells treated with different concentrations of the doxorubicin for 24 hours, FIG. 24B shows the analysis results of apoptosis of the 4T1 cells treated with different concentrations of the doxorubicin for 48 hours, and FIG. 24C shows the analysis results of apoptosis of the 4T1 cells treated with different concentrations of the doxorubicin for 72 hours. In FIG. 24A to FIG. 24C, “Ctrl” refers to the control group, “Cmax” refer to the maximum serum concentration of doxorubicin in clinically observation.

As shown in FIG. 24A to FIG. 24C, the 4T1 cells of 8×Cmax have the highest apoptosis level in Area 1 at 24 hours, 48 hours and 72 hours, which indicates that the concentration of the doxorubicin is the highest among all of the areas so as to lead the apoptosis in Area 1. Therefore, by controlling the circulation and the diffusion, the microenvironment-simulated cell culture system and the microenvironment-simulated cell culturing method of the present disclosure are capable of rapidly generating the spatial gradient microenvironment of the drug within a short period of time, and the survival condition of the 4T1 cells can be mimicked.

### IX. Analysis of the Effect of the Impermeable Materials of the Microenvironment-Simulated Cell Culture System

In the present test, two of the microenvironment-simulated cell culture systems of Example 4 are provided, wherein the first base plate, the upper base plate, the middle base plate and the lower base plate of one microenvironment-simulated cell culture system are made of the polyethylene terephthalate (“PET-based microenvironment-simulated cell culture system” hereafter), and the first base plate, the upper base plate, the middle base plate and the lower base plate of the other microenvironment-simulated cell culture system are made of the polydimethylsiloxane (“PDMS-based microenvironment-simulated cell culture system” hereafter). Then, the immune cells are seeded in the cell culture chamber of each of the microenvironment-simulated cell culture systems so as to observe the infiltration of the immune cells in different areas of each of the PET-based microenvironment-simulated cell culture system and the PDMS-based microenvironment-simulated cell culture system.

As shown in the results, the extent of immune cell infiltration differs among different areas and between the PET-based microenvironment-simulated cell culture system and the PDMS-based microenvironment-simulated cell culture system, wherein he PET-based microenvironment-simulated cell culture system with the dynamic condition results in a higher level of infiltration, the PDMS-based microenvironment-simulated cell culture system with the static condition exhibits an intermediate level of infiltration, and the PET-based microenvironment-simulated cell culture system with the static condition shows a lower level of infiltration. These differences are associated with the variations in oxygen diffusion and interstitial transport within the microenvironment-simulated cell culture system, which influence the migration and the spatial distribution of the immune cells across the constructed tissue in the cell culture chamber.

### X. Analysis of the Effects of the Seeding Density of Cancer Cells in the Cell Culture Chamber of the Microenvironment-Simulated Cell Culture System

In the present test, the microenvironment-simulated cell culture system of Example 4 is used, wherein the cancer cells are seeded in the cell culture chamber of the microenvironment-simulated cell culture system with different seeding densities for 48 hours. Further, the cancer cells used in the present test are the HEPG2 cells, the MIA-PaCa-2 cells, the SW620 cells, the WiDr cells, the HCT-116 cells and the MDA-MB-231 cells so as to observe the oxygenation profiles in the cell culture chamber of the microenvironment-simulated cell culture system. Further, the seeding number of the cancer cells is 1.25×10⁷ per milliliter, and the seeding density corresponding to 1.25×10⁷ of the cancer cells is set as 1×.

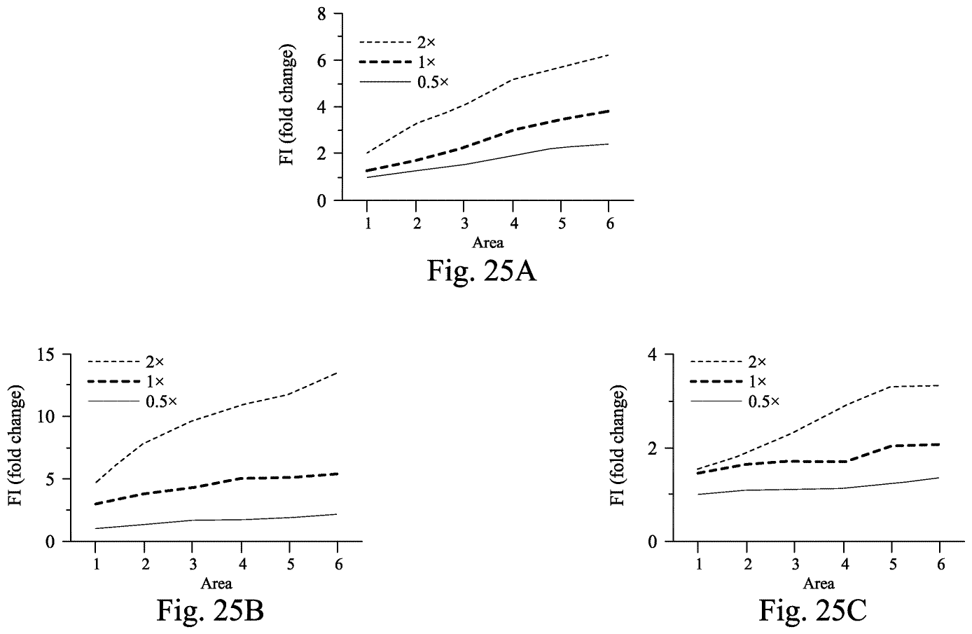

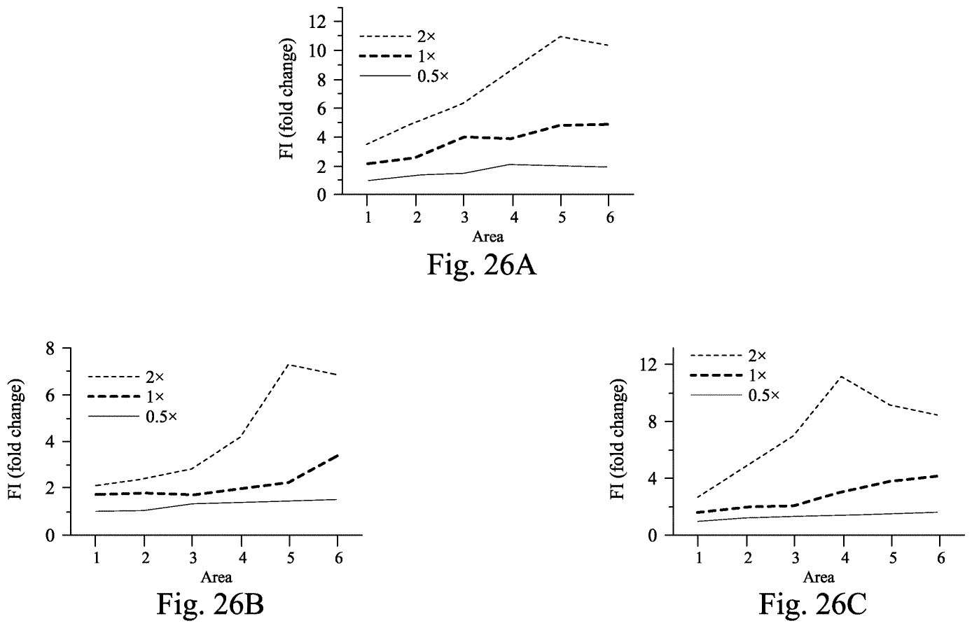

Reference is made to FIG. 25A to FIG. 26C, wherein FIG. 25A shows the oxygenation profiles of the HEPG2 cells in different areas of the cell culture chamber of the microenvironment-simulated cell culture system of Example 4 with different seeding densities, FIG. 25B shows the oxygenation profiles of the MIA-PaCa-2 cells in different areas of the cell culture chamber of the microenvironment-simulated cell culture system of Example 4 with different seeding densities, FIG. 25C shows the oxygenation profiles of the SW620 cells in different areas of the cell culture chamber of the microenvironment-simulated cell culture system of Example 4 with different seeding densities, FIG. 26A shows the oxygenation profiles of the WiDr cells in different areas of the cell culture chamber of the microenvironment-simulated cell culture system of Example 4 with different seeding densities, FIG. 26B shows the oxygenation profiles of the HCT-116 cells in different areas of the cell culture chamber of the microenvironment-simulated cell culture system of Example 4 with different seeding densities, and FIG. 26C shows the oxygenation profiles of the MDA-MB-231 cells in different areas of the cell culture chamber of the microenvironment-simulated cell culture system of Example 4 with different seeding densities. In FIG. 25A to FIG. 26C, “1×” refers to that the seeding number of the cancer cells is 1.25×10⁷ per milliliter, “0.5×” refers to that the seeding number of the cancer cells is 0.75×10⁷ per milliliter, and “2×” refers to that the seeding number of the cancer cells is 2.5×10⁷ per milliliter.

As shown in FIG. 25A to FIG. 26C, the increase in the cell density leads to enhanced oxygen consumption, thereby producing a more pronounced hypoxic gradient from the circulation-proximal region to the distal region of the cell culture chamber. Conversely, the lower cell density results in reduced oxygen consumption and a diminished hypoxic gradient.

Therefore, the density-dependent oxygen consumption enables tunable formation of distinct hypoxic microenvironments within the microenvironment-simulated cell culture system. By adjusting cell density, the degree and the spatial distribution of hypoxia can be precisely controlled, allowing the generation of different oxygenation states that mimic heterogeneous physiological or pathological conditions.

XI. Analysis of the Microenvironment-Simulated Cell Culture System Used to Culture the Cells from Multiple Cellular Sources

In the present test, the cancer cells derived from the pancreatic ductal adenocarcinoma tissue of mice (“the cancer cells of PDAC mice” hereafter), the colorectal cancer tissue of mice (“the cancer cells of CRC mice” hereafter) and the breast cancer tissue of mice (“the cancer cells of BC mice” hereafter) are respectively cultured in the cell culture chamber of the microenvironment-simulated cell culture system of Example 4. Then, the cancer cells of PDAC mice and the cancer cells of CRC mice are treated with the 5-FU and the oxaliplatin for 24 hours, and the cancer cells of BC mice are treated with the chloroquine, the cisplatin and the combination thereof for 24 hours. After the treatment, the cancer cells of PDAC mice, the cancer cells of CRC mice and the cancer cells of BC mice are stained with Caspase 3/7 so as to observe the fluorescence intensity in the cancer cells so as to measure the apoptosis level thereof. Further, a control group is also provided in the present test, wherein the control group is not treated with any chemotherapy drug, and the data of the cancer cells of PDAC mice, the cancer cells of CRC mice and the cancer cells of BC mice are respectively normalized to the control group.

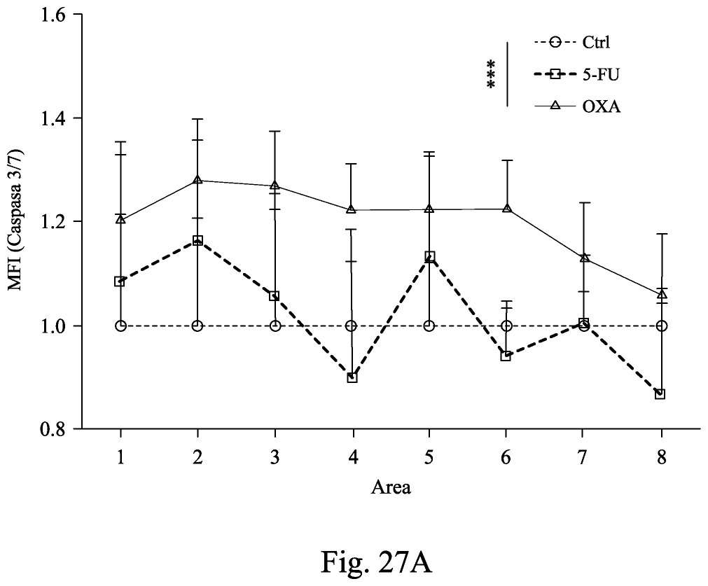

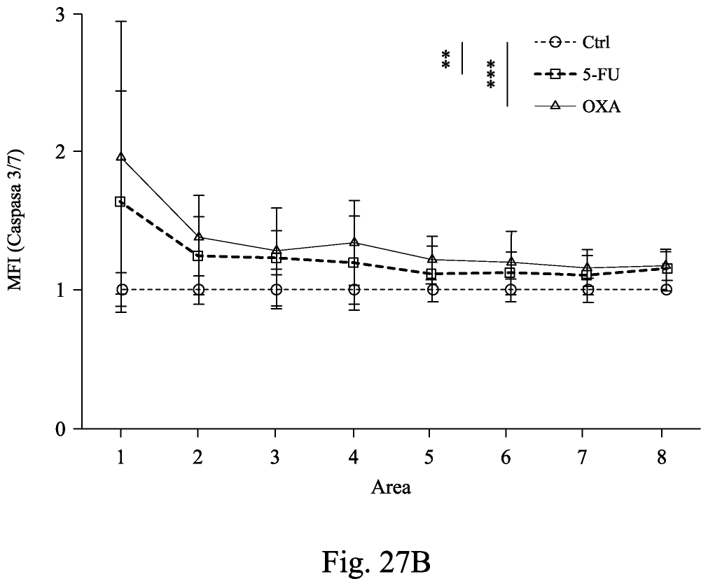

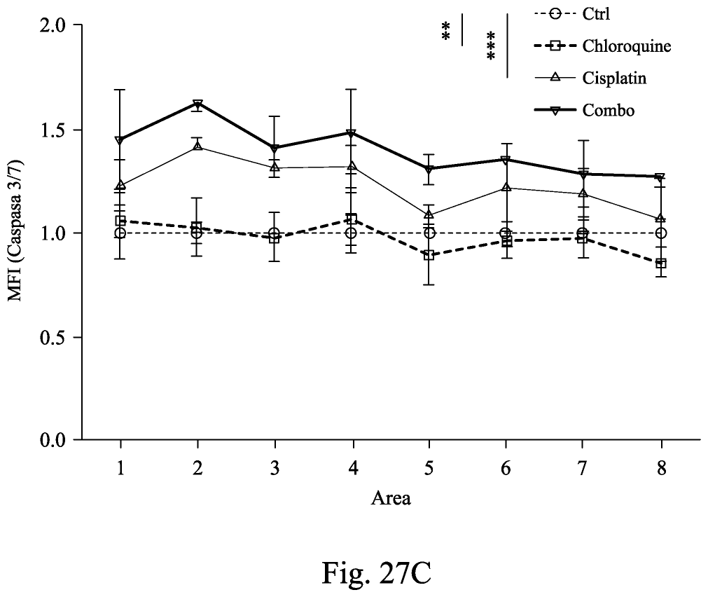

Reference is made to FIG. 27A to FIG. 27C, wherein FIG. 27A shows the signals of Caspase 3/7 of the cancer cells of PDAC mice in different areas of the cell culture chamber of the microenvironment-simulated cell culture system of Example 4, FIG. 27B shows the signals of Caspase 3/7 of the cancer cells of CRC mice in different areas of the cell culture chamber of the microenvironment-simulated cell culture system of Example 4, and FIG. 27C shows the signals of Caspase 3/7 of the cancer cells of BC mice in different areas of the cell culture chamber of the microenvironment-simulated cell culture system of Example 4. In FIG. 27A to FIG. 27C, “Ctrl” refers to the control group, “5-FU” refers to the cancer cells treated with the 5-FU, “OXA” refers to the cancer cells treated with the oxaliplatin, and “Combo” refers to the cancer cells treated with the chloroquine and the cisplatin simultaneously.

As shown in FIG. 27A to FIG. 27C, when the cancer cells derived from in vivo tumors, rather than cell lines, are cultured within the cell culture chamber of the microenvironment-simulated cell culture system of the present disclosure, the drug efficacy testing also can be conducted therein. Further, please refer to Table 1 and Table 2 as follows.

TABLE 1
breast cancer

| Gene | Tumor | Example 4 |
|---|---|---|
| HMBS | a | a |
| TP53 | a | a |
| STK11 | a | a |
| SMARCA4 | b | b |
| PIK3CA | c | N/A |

TABLE 2
liver cancer

| Gene | Tumor | Example 4 |
|---|---|---|
| KRAS | a | a |
| PIK3CA | a | a |
| AXIN1 | b | N/A |

In Table 1 and Table 2, “a” represents structural interaction variant, “b” represents stop gained, and “c” represents protein to protein contact. As shown in Table 1 and Table 2, when the tumor cells derived from in vivo tumors are cultured within the microenvironment-simulated cell culture system of Example 4 for 48 to 72 hours, the molecular characterization indicates that a substantial portion of high-impact genetic alterations present in the original tumor, such as structural variants, missense mutations, frameshift mutations, and splice region variants, may be retained following culture. These alterations may include, but are not limited to, structural variants, and the extent of retention may vary depending on tumor type, sample condition, and culture parameters.

Therefore, the microenvironment-simulated cell culture system of the present disclosure has broad market potential.

### XII. Analysis of the Microenvironment-Simulated Cell Culture System Used to Culture the Constructed Tissue

In the present test, the cancer cells (CD45⁻/PDPN⁻/CD326⁺) derived from in vivo sources are cultured under the dynamic condition in the cell culture chamber of the microenvironment-simulated cell culture system of Example 4, wherein the cancer cells will proliferate and then differentiate to from a constructed cancer tissue in the cell culture chamber. Then, the cells of the constructed cancer tissue are harvested from the cell culture chamber via the other one of the fluid delivery ports for analyzing the T cell infiltration.

Further, the T cell infiltration conditions of the tumoroid cultured in other system and the original tumor tissue are also compared with those of the constructed cancer tissue cultured in the cell culture chamber of the microenvironment-simulated cell culture system of Example 4 so as to illustrate the efficiency of the microenvironment-simulated cell culture system of the present disclosure used to culture the constructed tissue.

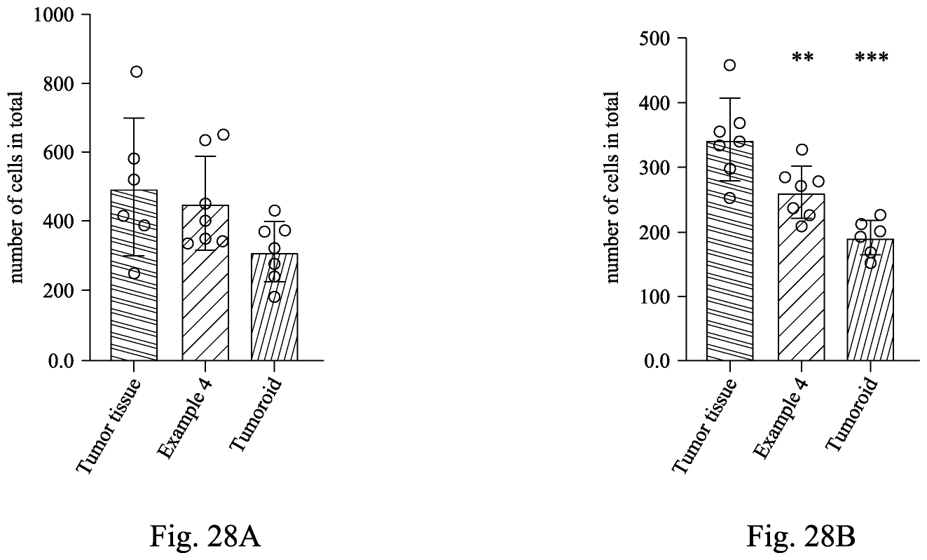

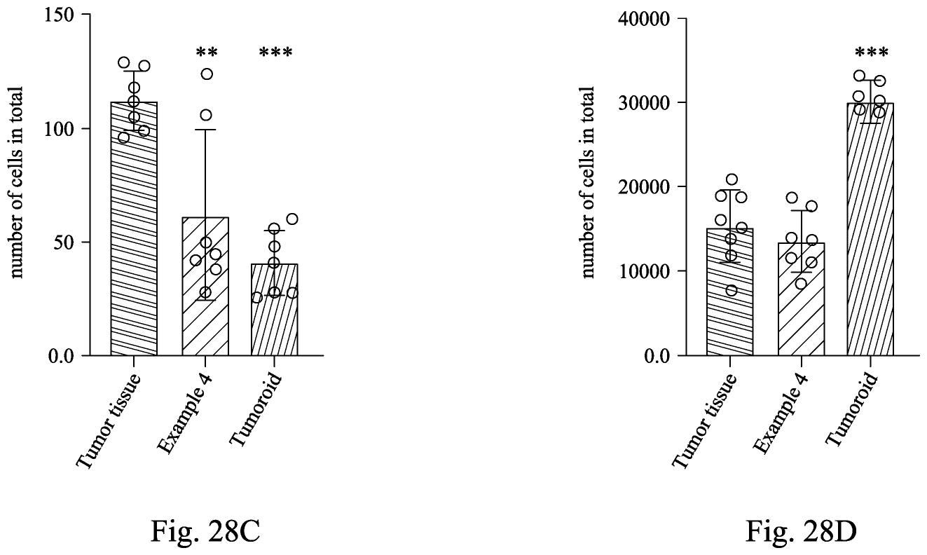

Reference is made to FIG. 28A to FIG. 28D, wherein FIG. 28A shows the numbers of the macrophages (CD45⁺, F4/80⁺) in the original tumor tissue, the constructed cancer tissue in the microenvironment-simulated cell culture system of Example 4 and the tumoroid, FIG. 28B shows the numbers of the CD3⁺/CD8⁺ T-cell in the original tumor tissue, the constructed cancer tissue in the microenvironment-simulated cell culture system of Example 4 and the tumoroid, FIG. 28C shows the numbers of the CD3⁺/CD4⁺ T-cell in the original tumor tissue, the constructed cancer tissue in the microenvironment-simulated cell culture system of Example 4 and the tumoroid, and FIG. 28D shows the numbers of the cancer cell in the original tumor tissue, the constructed cancer tissue in microenvironment-simulated cell culture system of Example 4 and the tumoroid.

As shown in FIG. 28A to FIG. 28C, compared to the tumoroid, the numbers of the macrophages, the CD3⁺/CD8⁺ T-cell and the CD3⁺/CD4⁺ T-cell of the constructed cancer tissue in the microenvironment-simulated cell culture system of Example 4 are more likely to be those of the original tumor tissue. Further, as shown in FIG. 28D, the number of the cancer cell in the tumoroid is more than those of the original tumor tissue and the constructed cancer tissue in the microenvironment-simulated cell culture system of Example 4. Hence, the constructed cancer tissue in the microenvironment-simulated cell culture system of the present disclosure more closely recapitulates the biological properties of the original tumor tissue.

### XIII. Analysis of the Effects of the Seeding Density of Mammalian Cells in the Cell Culture Chamber of the Microenvironment-Simulated Cell Culture System

In the present test, the microenvironment-simulated cell culture system of Example 4 is used, wherein the AML-12 liver cells are seeded in the cell culture chamber of the microenvironment-simulated cell culture system with different seeding densities for 48 hours, wherein the seeding densities of the AML-12 liver cells are 3.13×10⁶ cells/mL, 6.25×10⁶ cells/mL and 1.25×10⁷ cells/mL, respectively.

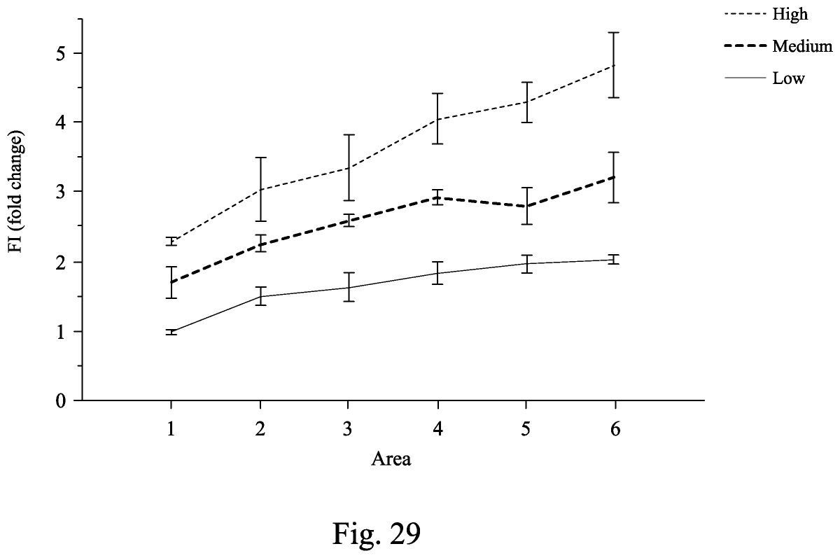

Reference is made to FIG. 29, which shows the oxygenation profiles of the AML-12 liver cells in different areas of the cell culture chamber of the microenvironment-simulated cell culture system of Example 4 with different seeding densities. In FIG. 29, “High” represents the seeding density of the AML-12 liver cells is 1.25×10⁷ cells/mL, “Medium” represents the seeding density of the AML-12 liver cells is 6.25×10⁶ cells/mL, and “Low” represents the seeding density of the AML-12 liver cells is 3.13×10⁶ cells/mL. As shown in FIG. 29, when the seeding density of the AML-12 liver cells ranges from 3.13×10⁶ cells/mL to 1.25×10⁷ cells/mL, the density-dependent oxygen consumption enables tunable formation of distinct hypoxic microenvironments within the microenvironment-simulated cell culture system. Further, when the cell density reaches a specific range, an oxygen concentration range of approximately 12% to 20%, which is important in maintaining the cell function and mimicking the physiological microenvironment, can be established within the cell culture chamber.

### XIV. Analysis of the Microenvironment-Simulated Cell Culture System Used to Co-Culture the Cancer Cells and Immune Cells

In the present test, the cancer cells are co-cultured with the PBMC (peripheral blood mononuclear cell) in the cell culture chamber of the microenvironment-simulated cell culture system of Example 4, wherein the seeding number of the cancer cells is 1.25×10⁷ per milliliter, and the mixing ratio of the PBMC is 5% to 20% of the cancer cells so as to simulate the presence of tumor-infiltrating lymphocytes within the tumor tissue. Further, the cancer cells are treated with the pembrolizumab so as to observe the survival state of the cancer cells.

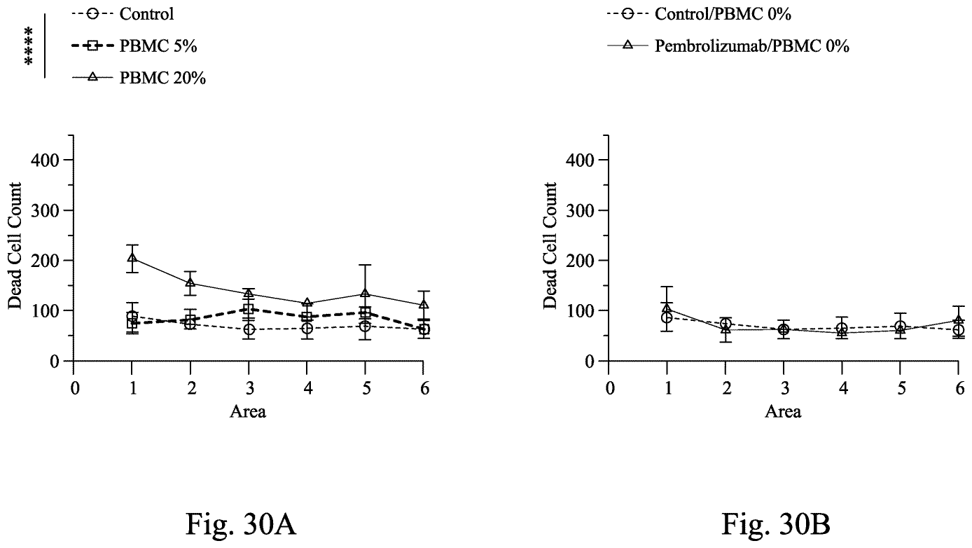

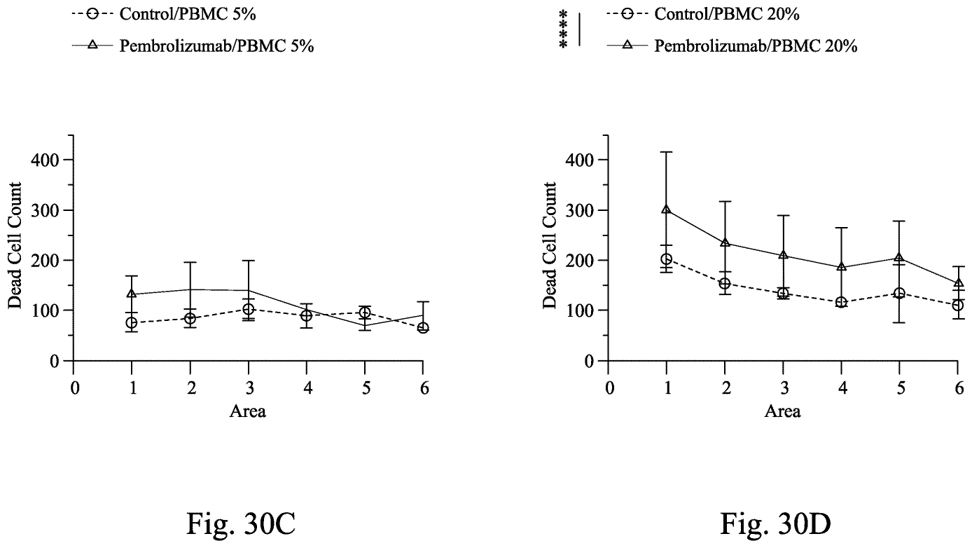

Reference is made to FIG. 30A to FIG. 30D, wherein FIG. 30A shows the dead cell count result of the cancer cells co-cultured with the PBMC in different areas of the cell culture chamber of the microenvironment-simulated cell culture system of Example 4, FIG. 30B shows the dead cell count result of the cancer cells treated with 0% of the pembrolizumab in different areas of the cell culture chamber of the microenvironment-simulated cell culture system of Example 4, FIG. 30C shows the dead cell count result of the cancer cells co-cultured with 5% PBMC and treated with the pembrolizumab in different areas of the cell culture chamber of the microenvironment-simulated cell culture system of Example 4, and FIG. 30D shows the dead cell count result of the cancer cells co-cultured with 20% PBMC and treated with the pembrolizumab in different areas of the cell culture chamber of the microenvironment-simulated cell culture system of Example 4.

As shown in FIG. 30A, when the cancer cells are co-cultured with the PBMC without any drug, the dead cell number of the cancer cells decreases along from Area 1 to Area 6. Further, the dead cell number of the cancer cells co-cultured with 20% PBMC is larger than that of the 5% PBMC. As shown in FIG. 30B, when the cancer cells are treated with the pembrolizumab without the PBMC, the dead cell number of the cancer cells also decreases along from Area 1 to Area 6. Furthermore, as shown in FIG. 30C and FIG. 30D, when the cancer cells co-cultured with 5% and 20% PBMC along with the pembrolizumab, the dead cell number of the cancer cells decreases along from Area 1 to Area 6.

Accordingly, although the density of the PBMC remained consistent across regions, the cancer cells response exhibited differential distribution across different areas, and this suggests that the immune response may be influenced by fluid transport conditions and drug distribution, forming a gradient of efficacy and function that varies along spatial locations.

To sum up, in the microenvironment-simulated cell culture system and the microenvironment-simulated cell culturing method of the present disclosure, not only the cells can be cultured in the three-dimensional space of the cell culture chamber so as to simulate the hypoxia condition of the area far away from the circulatory system in the tumor for drug screening or immunoassay testing, but also the states that the tumor is rich in the extracellular matrix and the hyperplasia of the connective tissues can be simulated by supplying the extracellular matrix fluids such as collagen. Therefore, the microenvironment-simulated cell culture system of the present disclosure can be used for anticancer drug screening and related treatment tests for different types of cancer, and has clinical application potential.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure covers modifications and variations of this disclosure provided they fall within the scope of the following claims.

What is claimed is:

1. A microenvironment-simulated cell culture system, comprising:
a cell culture chip, comprising:
a mainbody;
a cell culture chamber disposed in the mainbody and comprising a first side portion and a second side portion, wherein the first side portion and the second side portion are respectively disposed on two ends of the cell culture chamber along a long axis of the mainbody;
two fluid delivery ports separately disposed on the mainbody and respectively connected to the cell culture chamber; and
a sample loading well disposed on the mainbody and connected to the cell culture chamber;
a fluid storage device pipe-connected to the cell culture chip, wherein the fluid storage device is connected to the cell culture chamber by one of the fluid delivery ports; and
a fluid driving member pipe-connected to the fluid storage device and the cell culture chip, wherein the fluid driving member is connected to the cell culture chamber by the other one of the fluid delivery ports;
wherein the cell culture chamber is substantially a long-stripped slot, two long sides of the cell culture chamber are parallel to the long axis of the mainbody, a length ratio of a short side of the cell culture chamber to one of the long sides of the cell culture chamber is 1:1 to 1:4, and a length of the one of the long sides of the cell culture chamber is 1.3 cm to 2.0 cm.

2. The microenvironment-simulated cell culture system of claim 1, wherein:
the cell culture chip is a multi-layers structure and comprises a first base plate, a second base plate, a third base plate, a fourth base plate, a fifth base plate, a sixth base plate and a seventh base plate;
the first base plate has a first surface, and the two fluid delivery ports are separately opened on the first surface, wherein the first base plate, the second base plate, the third base plate and the fifth base plate are stacked in sequence to form a fluid channel, and the fluid channel is connected to the two fluid delivery ports and the cell culture chamber;
the fourth base plate has a second surface, and the sample loading well is opened on the second surface, wherein the fourth base plate and the fifth base plate are stacked in sequence to form a loading channel, and the loading channel is connected to the sample loading well and the cell culture chamber; and
the fifth base plate, the sixth base plate and the seventh base plate are stacked in sequence to form the cell culture chamber.

3. The microenvironment-simulated cell culture system of claim 2, wherein:
the first base plate, the second base plate and the third base plate are stacked in sequence to form a first covering unit, and the first covering unit covers the first side portion; and
the fourth base plate covers the second side portion.

4. The microenvironment-simulated cell culture system of claim 2, wherein the first base plate, the second base plate, the third base plate, the fourth base plate, the fifth base plate, the sixth base plate and the seventh base plate are made of an impermeable material.

5. The microenvironment-simulated cell culture system of claim 4, wherein the impermeable material is polydimethylsiloxane, poly(methyl methacrylate), polyethylene terephthalate, acrylic, polycarbonate, polystyrene, silicone rubber or glass, and the impermeable material is a transparent material.

6. The microenvironment-simulated cell culture system of claim 1, wherein the two fluid delivery ports are disposed along a direction parallel to the short side of the cell culture chamber.

7. The microenvironment-simulated cell culture system of claim 1, wherein the fluid driving member is a peristaltic pump.

8. The microenvironment-simulated cell culture system of claim 1, wherein the length ratio of the short side of the cell culture chamber to the one of the long sides of the cell culture chamber is 1:2.

9. The microenvironment-simulated cell culture system of claim 1, wherein a height of the cell culture chamber perpendicular to the long axis of the mainbody is 180 μm to 220 μm.

10. The microenvironment-simulated cell culture system of claim 1, wherein a length of the short side of the cell culture chamber is 0.5 cm to 1.0 cm.

11. A microenvironment-simulated cell culture system, comprising:
a cell culture chip, comprising:
at least one cell culture chamber, wherein the at least one cell culture chamber comprises a first side portion and a second side portion, and the first side portion and the second side portion are respectively disposed on two ends of the at least one cell culture chamber along a long axis of the cell culture chip;
at least one fluid storing well connected to the at least one cell culture chamber;
at least three membrane pump units connected to the at least one fluid storing well, wherein the at least three membrane pump units are arranged in sequence;
at least one air transporting channel connected among the at least three membrane pump units, wherein the at least one air transporting channel is substantially s-shaped, and a width of the air transporting channel is 0.06 mm to 0.12 mm;
at least one valve connected to the air transporting channel; and
at least two air transporting pores, wherein one of the at least two air transporting pores is connected to the at least three membrane pump units, and the other one of the at least two air transporting pores is connected to the at least one valve;
a fluid storage device connected to the at least one fluid storing well, wherein the fluid storage device is for supplying a cell culture medium to the at least one fluid storing well; and
an air pump connected to the at least two air transporting pores, wherein the at least one valve is connected between the air transporting channel and the air pump;
wherein the at least one cell culture chamber is substantially a long-stripped slot, two long sides of the at least one cell culture chamber are parallel to the long axis of the cell culture chip, a length ratio of a short side of the at least one cell culture chamber to one of the long sides of the at least one cell culture chamber is 1:1 to 1:4, and a length of the one of the long sides of the at least one cell culture chamber is 1.3 cm to 2.0 cm.

12. The microenvironment-simulated cell culture system of claim 11, wherein the cell culture chip is composed of a first base plate, an upper base plate, a middle base plate and a lower base plate, the first base plate, the upper base plate, the middle base plate and the lower base plate are stacked in sequence, and the upper base plate, the middle base plate and the lower base plate are made of an impermeable material.

13. The microenvironment-simulated cell culture system of claim 12, wherein the impermeable material is polydimethylsiloxane, poly (methyl methacrylate), polyethylene terephthalate, acrylic, polycarbonate, polystyrene, silicone rubber or glass.

14. A microenvironment-simulated cell culturing method, comprising:
providing the microenvironment-simulated cell culture system of claim 11;
seeding a cell in the at least one cell culture chamber via the at least one fluid storing well, wherein the air pump drives the at least three membrane pump units in sequence to transport the cell culture medium comprising the cell in the at least one fluid storing well to the at least one cell culture chamber, and a number of the cell seeded in the at least one cell culture chamber is 0.25×10⁶ to 2.5×10⁷;
incubating the cell for a reaction time and then detecting a biochemical condition of the cell so as to obtain a microenvironment-simulated cell culturing result;
wherein a positive pressure of the air pump is 3 to 12 psi, a negative pressure of the air pump is −3 to −12 psi, and a flow rate of the cell culture medium driven by the air pump is 30 μL/min to 60 μL/min.

15. The microenvironment-simulated cell culturing method of claim 14, wherein the cell comprises a mammalian cell and at least one cancer cell.

16. The microenvironment-simulated cell culturing method of claim 15, wherein the at least one cancer cell is derived from a live tumor tissue.

17. The microenvironment-simulated cell culturing method of claim 15, wherein the mammalian cell comprises a fibroblast and an immune cell.
